(19) United States
(12) Patent Application Publication
(10) Pub. No.: US 2003/0055106 A1
(43) Pub. Date: Mar. 20, 2003

(54) Therapeutic uses of peroxometallic compounds

(76) Inventors: Robert Faure, Sillery (CA); Pierre Savard, Levis (CA); Charles Doillon, Montchatel (CA); Bruno Joseph Battistini, St. Nicolas (CA); Martin Olivier, Sillery (CA); Barry Posner, Westmount (CA)

Correspondence Address: NILLES & NILLES, S.C., FIRSTAR CENTER, SUITE 2000, 777 EAST WISCONSIN AVENUE, MILWAUKEE, WI, 53202-5345, US

(21) Appl. No.: 10/150,756

(22) Filed: May 17, 2002

Related U.S. Application Data
(63) Continuation-in-part of application No. 09/631,804, filed on Aug. 3, 2000.

(30) Foreign Application Priority Data
Aug. 12, 1999 (CA) 2.280.249

(51) Int. Cl. A61K 31/555; A61K 31/28

(52) U.S. Cl. 514/492

(57) ABSTRACT

The present invention relates to the use of peroxometallic compounds, such peroxovanadium compounds, to prevent angiogenesis, restenosis and the production of endothelins, as immunomodulators and as antitumorigenic agents. Peroxometallic compounds are preferred since they are more potent, and less toxic, than their “oxo” counterparts, as exemplified by specific peroxovanadium compounds. Anti-angiogenic activity was verified in vitro against human umbilical vascular endothelial cells (HUVECs) as well as ex ovo using the chicken chorioallantoic assay membrane and in the rat aortic ring model and a Matrigel assay in vivo. Peroxovanadium compounds also decrease basal levels and inhibit the increase in plasma endothelins occurring following insulin induction in rats. It is proposed that peroxovanadium compounds are therapeutically-active anti-angiogenics and useful in preventing vascular restenosis by acting, inter alia, by inhibiting one or several protein tyrosine phosphatases (PTPs) involved in the proliferation, differentiation and migration of cells or the secretion of peptides (such endothelins and immunomodulators), or both. These compounds have also been found to be suitable as antitumorigenic agents in the treatment of cancer, such as breast cancer and prostate cancer.

### FIELD OF THE INVENTION

This invention relates to the use of peroxometallic compounds, such as potassium bisperoxo(1,10-phenanthroline)oxovanadate [bpV(phen)], potassium bisperoxo(pyridine-2-carboxylato)oxovanadate [bpV(pic)] and potassium bisperoxo(2,2′-bipyridyl) oxovanadate [bpV(bipy)], for preventing angiogenesis, restenosis and the production of endothelins, and as adjuvants for vaccination. The compounds of the present invention are also suitable as antitumorigenic agents; for example, experimental results reveal that they are efficacious in the treatment of breast cancer and prostate cancer.

### BACKGROUND OF THE INVENTION

A) Peroxovanadium Compounds:

Synthetic peroxovanadium (pV) compounds are structurally versatile molecules which are potent inhibitors of phosphotyrosyl phosphatases (PTPs) (1). These compounds contain one oxo ligand, one or two peroxo groups, one ancillary ligand, all coordinated to vanadium. They are stable in aqueous solution at physiological pH when shielded from light. pVs are cytostatic agents, unlike many other antitumor molecules. Their mode of action lies in the modulation of the activity of cellular transduction pathways involved in the progression of pathological conditions. Their effects are transitory and disappear within a few days after administration(2).

Phosphotyrosine phosphatases (PTPs) are enzymes which remove phosphates from tyrosine residues of proteins. They are involved in several cell functions regulating proliferation, differentiation and metabolism. Their number is estimated at about 100 in the human genome (3). These enzymes function by engulfing in their catalytic site phosphates located on the tyrosine residues of target proteins. The mechanisms underlying the inhibition of PTPs and the specificity of these peroxo-anionic compounds have been characterized. The inhibiting potential of PTPs by pVs is a 100 to a 1000 times more powerful than that of oxovanadate (1). When compared to known inhibitors of PTPs such as orthovanadate, molybdate, tungstate and zinc, the increased inhibiting potential of pV can be explained by the presence of the peroxide groups, which have the ability to irreversibly oxidize an essential conserved cysteine residue located in the catalytic domain of practically all PTPs (4).

The possibility of manipulating the ancillary ligands of pVs is important in regulating potency and specificity (1). The ancillary ligands are more or less hydrophilic or hydrophobic and provide the molecule with a specific mode of action and distribution for the different PTPs. These ligands allow for the specific targeting of certain PTPs.

B) Angiogenesis:

Almost all tissues and organs develop a vascular network which provides cells with nutrients and oxygen and enables the elimination of metabolic waste. Once formed, the vascular network is a stable system with a slow rate of cellular turn over (5) and yet, the endothelium can become one of the most rapidly proliferating of all cell types when stimulated (6). Indeed, the formation of new blood vessels can cause serious physiological complications. For example, while cornea and cartilage are avascular in healthy situations, several diseases involving these tissues are complicated by the massive arrival of blood vessels. Eye angiogenic diseases include neovascular glaucoma, retrolental fibroplasia, macular degeneration and neovascularization of corneal grafts. Joint angiogenic diseases include rheumatoid arthritis and arthrosis. Psoriasis, a chronic condition of the skin, also exhibits hypervascularization at the surface of the skin. Finally, solid tumor growth is critically dependent upon the formation of new blood vessels to progress locally and spread all over the body (7,8). The maintenance of existing blood vessels also requires the regulation of cell replication and/or differentiation. For example, acute and chronic pathological processes, such as atherosclerosis, post-angioplastic restenosis and hypertension, involve the proliferation of different cellular components of mature blood vessels (endothelial cells, smooth muscle cells, myocytes and fibroblasts).

Several factors, including certain peptides and proteins, can induce a vascular response in vivo. They are endogenous substances, such as EGF/TGF-α (Epidermal Growth Factor/Transforming Growth Factor-alpha), TGF-β (Transforming Growth Factor-beta), TNF-α (Tumor Necrosis Factor-alpha), angiogenin, prostaglandine E₂, and monobutyrine (9-16). However, these factors have almost no mitogenic effect on endothelial cells in culture (TGF-α, EGF, angiogenine, prostaglandine E₂, monobutyrine) and, paradoxically, inhibit their growth (TGF-β, TNF-α) (10-17). Their angiogenic action is thus indirect, depending for the most part upon the inflammatory response (17). Inflammatory cells produce some factors, such as aFGF (acidic Fibroblast Growth Factor), bFGF (basic Fibroblast Growth Factor), PDGF (Platelet-Derived Growth Factor), and VEGF (Vascular Endothelial Growth Factor), which are capable of stimulating the proliferation of endothelial cells in vitro and angiogenesis in vivo (17-27).

The angiogenic process, as currently understood, can be summarized as follows: a cell activated by a mutation, lack of oxygen, etc, releases angiogenic molecules (28-33) that attract inflammatory and endothelial cells and promote their proliferation. Following the binding of leukocytes to vascular endothelial cells, the latter reorganize the protein arrangements on their membranes to activate the angiogenic process (34, 35). During migration to the target tissue, inflammatory cells release substances that intensify the angiogenic effect (36, 37). Activated vascular endothelial cells respond to the angiogenic signal by secreting proteases which digest blood vessel walls to enable migration toward the target tissue (38-40). Several protein fragments produced by the digestion of the blood-vessel walls intensify the proliferative and migratory activity of the endothelial cells (41-43). Finally, the endothelial cells rearrange their adhesive membrane proteins to generate the formation of capillary tubes.

Angiogenesis is thus a complex process consisting of several critical cellular events (44-46), among which the following may be readily identified:

binding of leukocytes to endothelial cells and induction;

migration of inflammatory cells to the target tissue;

regression of the pericytes of the existing vascular system;

dissolution of blood-vessel walls by proteases;

endothelial cell migration;

endothelial cell proliferation;

endothelial cell differentiation and arrangement into a tubular shape;

formation of the capillary network;

anastomosis; and

initiation of blood flow.

Agents that are known to induce the proliferation of endothelial cells include sodium orthovanadate (47). The mechanism by which this agent induces an invasive phenotype in capillary endothelial cells is not clearly understood. However, the effects of vanadate on cultured cells are similar, in many respects, to those elicited by PMA (48), bFGF (49), and certain retroviral transforming proteins, which act by inducing tyrosine-specific protein phosphorylation (50-53). Consistent with these observations is the finding that both phorbol ester and various growth factors including bFGF, VEGF and PDGF stimulate the phosphorylation of cellular proteins on tyrosine residues whereas vanadate, perhaps by inhibiting tyrosine phosphatase(s), produces a marked increase in tyrosine phosphorylation.

pVs were expected to promote endothelial cell proliferation, like vanadate. On the contrary, these agents have been shown to inhibit the proliferation of several cell types (2). In some cases, the cells are arrested at G2/M. The simplest hypothesis to explain this restriction is that PTPs controlling cell mitosis are targets for bpV(phen) and pbV (pic) inhibitors. The cdc25 protein was proposed as one candidate target for pV compounds (2).

International Patent Publication WO 95/19177 teaches the use of vanadate compounds for the treatment of proliferative disorders, metastasis and drug-resistant tumors. Although these vanadate compounds are stated to be anti-proliferative and anti-collagenolytic, no indication of any anti-angiogenic activity has been ascribed to them. This publication further shows that an anti-tumor effect is observed at dosages of vanadate higher than 5 mM. It is admitted that a concentration of vanadate compound of 1.3 mM or lower has no apparent anti-tumor effect.

Montesano et al. (47) teach, on the contrary, that vanadate compounds cause endothelial cells to proliferate. Hence, their findings would indicate that these compounds are pro-angiogenic and not anti-angiogenic.

U.S. Pat. No. 5,716,981 (Hunter et al.) mentions the use of vanadium compounds, namely oxovanadate, orthovanadate and vanadyl compounds, in anti-angiogenic applications. However, this reference provides no experimental evidence to substantiate the anti-angiogenic effects of these compounds. The compounds are stated to be possibly equivalent to Paclitaxel (an anti-angiogenic compound described in detail). Since the remaining body of the prior art suggests that vanadium compounds are pro-angiogenic, there is no enabling teaching in this patent to validate the use of these compounds as anti-angiogenics.

pVs are more powerful anti-tumor molecules than oxo compounds, such as vanadate. The direct antiproliferative activity of pVs on transformed cells is known (2). At concentrations found to be ineffective for vanadate compounds in International Patent Publication WO 95/19177, pV compounds are efficacious anti-tumor compounds.

Although the anti-tumor activity ascribed to cytotoxicity of vanadium compounds is known, their anti-angiogenic activity has not heretofore been disclosed in relation to either oxo or peroxo derivatives thereof.

C) Endothelins:

Endothelins (ETs), a family of three isopeptides, acting as important regulators of the physiological state of mature blood vessels. They are the most potent vasoconstrictors identified to date. In addition, they are known to stimulate the proliferation of endothelial cells, smooth muscles, myocytes and fibroblasts, as well as the synthesis of various growth factors, including VEGF (54, 55).

ETs are also considered to be angiogenic factors involved in tumor development (56). Most tumor cells synthesize and secrete ETs (57-61). Patients affected by different cancers show elevated blood concentrations of ETs (62-64). A reduction in the expression of ET receptor type B (RET_{B}) decreases the growth of tumor cells incubated in the presence of ETs (64). As indicated above, ETs promote proliferation and migration of endothelial cells (65). Expression of ET ligands and of ET receptors (RET_{A} and RET_{B}) was observed in the endothelial cells of a plurality of tumors (66, 67). ETs act in an autocrine fashion, promoting local angiogenesis (67). ETs are further involved in hemodynamic changes that go along with metastatic development. For example, the ratio of arterial hepatic blood flow to portal vein blood flow is abnormally high in patients having hepatic metastasis from colorectal tumors (69). This high blood flow ratio is due to the presence of a humoral mediator as demonstrated in vivo (70).

A tumoral vascular bed has no innervation and consequently does not respond to vasoconstrictive drugs. However, these drugs decrease normal hepatic blood flow and increase blood flow in tumors. Since ETs are potent vasoconstrictive and angiogenic factors involved in vascular remodeling and tumor development, they may be responsible for these altered hemodynamics. An ET inhibitor may therefore be a valuable tool for controlling intratumor blood flow and for influencing the growth and degeneration of tumors.

D) Angioplasty:

ETs are also known to stimulate the production of extracellular matrices by endothelial cells (71, 72). That is frequently observed upon vascular reconstruction or angioplasty (73, 74). This effect is particularly deleterious following vascular trauma due to restenosis. In 30-50% of patients, restenosis is characterized by the reexpansion of atherosclerotic lesions. The causes of this vascular disorder are due to a local vascular blockage caused by cell proliferation, cell migration and extracellular matrix production. Recently, ETs have been shown to be major players in vascular remodelling, particularly in such conditions as long term atherosclerosis, cardiac hypertrophy (congestive heart failure), hypertension (pulmonary and other), renal problems and certain systemic dysfunctions (75-79). ETs are also strongly involved in coronary and brain vasospasm leading to ischemia and a reduced rate of survival (80, 81). Thus, because of the potent vasoconstrictor and nitogenic activities of ETs, inhibitors of ET production would be expected to be generally useful as anti-hypertensive and anti-proliferative agents, and particularly useful prior to, during and after vascular surgery.

E) Immune Response:

To develop an effective cell-mediated immune response, infected macrophages (MØ) must be able to induce T lymphocyte activation in a specific manner. In turn, T cells, by secreting several lymphokines, can activate MØ for cytocidal functions (82, 84). Several investigators have demonstrated the importance of cytokines in the control of Leishmania infection (85-90). Thus, the integrity of MØ-T lymphocyte interactions is a primordial step for the immune response development, and MØ participation is crucial to its initiation and support. Tyrosine phosphorylation is a common event in the initiation of cell proliferation, and its role in signal transduction regulating cellular functions of nonproliferative haematopoietic cells is also well documented (91-97). We have recently demonstrated the importance of PTPs in NO regulation (98). Correlation between MØ PTP inhibition and an enhancement of NO production was supported by an increase in MØ PTK activity and tyrosyl residue hyperphosphorylation (78).

It has been demonstrated that the modulation of protein tyrosyl phosphorylation states will, in many cases, result in better responses from some cells to extracellular stimuli (98, 99-101). PTP can play a crucial role in the negative regulation of signal transduction culminating in T-lymphocyte activation. Prior studies have shown that pervanadate (a mixture of vanadate and hydrogen peroxide) stimulates transcription of the c-fos gene and accumulation of its mRNA as well as the expression of CD69 antigen and CD25 (101). Also, it was demonstrated that bpV(phen) could induce nucleus NF-kB translocation in T lymphocyte (102) and increase MØ iNOS mRNA expression (98). Rat kupffer cells in culture stimulated with platelet-activating factor and vanadate have shown a time- and concentration-dependent increase in phosphotyrosine in several proteins and of Prostaglandin E2 generation (99). As mentioned earlier, we recently demonstrated that MØ pretreated with bpV(phen) were more responsive to IFNγ stimulation as reflected by the greater amount of NO produced in comparison to Vanadate-treated and untreated cells (98). Although there is evidence that suggests that bpv(phen) can be a powerful activator of some immune functions, the capacity of either oxo or peroxo derivatives to induce cytokines (e.g. IFN(, IL-12 and IL-1) and chemokines (e.g. RANTES, MIP-1a,β, MIP-2, IP-10, MCP-1) recognized for their pivotal role in inflammatory response and the development of an effective immune response toward a specific antigen, and thus acting as an adjuvant, has not heretofore been disclosed.

### SUMMARY OF THE INVENTION

The present invention provides peroxometallic compounds, such as (pVs), that are useful against angiogenesis, restenosis and endothelin production, and as immunomodulators. They have also been found to be suitable for preventing further growth of established tumors. These molecules comprise a transition metal (such as vanadium, molybdenum, tungsten) and one oxo or peroxo groups. Molecules comprising peroxo groups are the more potent anti-angiogenic molecules. Preferably, the molecules also contain an ancillary ligand, which includes any molecule capable of binding the transition metal atom (usually, through bonds involving oxygen and nitrogen). Phenanthroline, picolinic acid, bipyridine, oxalic acid, 4,7-dimethyl-phenanthroline and peptides are examples of such ligands.

All these molecules can be used to inhibit the formation of new blood vessels and/or control systemic and local levels of endothelins (ETs) in the reparation of existing blood vessels.

The molecules containing peroxo anions are more powerful than their oxo counterparts. Therefore, the former can be used at much lower concentrations to reduce the toxicity that results from overexposure to transition metals (2).

Oxo transition metal complexes include oxo complexes such as vanadate, tungstate, molybdate, and vanadyl complexes, such as the following: methavanadate (VO₃⁻), orthovanadate (VO₄³⁻), salts thereof, vanadyl compounds (VO²⁺) like vanadyl acetyl acetonate and vanadyl sulfate. Similar complexes exist for other transition metals. Other suitable tungsten and molybdenum complexes include hydroxo derivatives derived from glycerol, tartaric acid and sugars, for example. The peroxo transition metal complexes include any oxidizing agent capable of combining with the transition metal. As such, the preferred peroxides comprise the following: t-butylhydroperoxide, benzoyl peroxide, m-chloroperoxibenzoic acid, cumene hydroperoxide, peracetic acid, hydroperoxiloneic acid, ethyl peroxide, pyridine peroxide and hydrogen peroxide.

The general structure of the compounds of the present invention is the following:

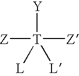

wherein:

T is a transition metal selected from the group consisting of vanadium, molybdenum, tungsten;

Y is oxygen or hydroxyl;

Z and Z′ are independently selected from oxygen and peroxide and at least one of them is peroxide; and

L and L′ are any group which can donate an electron pair.

In a preferred embodiment, the transition metal T is vanadium, Y is oxygen, Z and Z′ are peroxide and L and L′ are the nitrogen atoms of 1,10-phenanthroline.

In another preferred embodiment, the transition metal T is vanadium, Y is oxygen, Z and Z′ are peroxide and L and L′ are nitrogen or oxygen atoms of picolinic acid.

In yet another preferred embodiment, the transition metal T is vanadium, Y is oxygen, Z and Z′ are peroxide and L and L′ are nitrogen atoms of 2,2′-bipyridine.

The above pV compounds are potent anti-angiogenics, since they inhibit endothelial cell proliferation. They further inhibit neovascularization and the production of endothelins.

Moreover, administration of the pV compound bpv(bipy) in the rat model revealed a significant reduction in the degree of post-angioplastic vascular remodeling of the carotid artery. Additionally, the pV compound bpV(phen) was found to be a potent immunomodulator based on its capacity to induce cytokine and chemokine gene expression, to enhance cellular recruitment in response to an agonist and consequently act as an adjuvant in the context of vaccination.

In one specific embodiment, the present invention relates to the inhibiting action on tumor growth of bpV(phen), demonstrating efficiency in vivo. In addition it is shown that bpV(phen) has also the capacity to inhibit the migration of tumor cells in vitro.

Other objects, advantages and features of the present invention will become apparent upon reading the following non-restrictive description of preferred embodiments thereof, with references to the accompanying drawings.

### DESCRIPTION OF THE PREFERRED EMBODIMENTS OF THE PRESENT INVENTION

This invention will now be described by referring to specific embodiments and the appended Figures, which purpose is to illustrate the invention rather than to limit its scope.

### BRIEF DESCRIPTION OF THE FIGURES

FIG. 1: Peroxovanadium compounds inhibit the proliferation of endothelial cells. Human umbilical vein endothelial cells (HUVECs) were extracted with collagenase-controlled digestion. Pure HUVECs were used before the fourth passage (trypsin-EDTA at each passage). The cells were analyzed for their capacity to incorporate di-acetyl LDL and to be labelled with factor VIII-related antigen. Endothelial cells were plated at a density of 2500 cells/cm2 in a sterile plate coated with gelatin. Cells were cultured in complete medium (M199: heparin (90 mg/ml) or L-glutamine (2 mM), bicarbonate, FBS (20%) and ECGS (100 mg/ml) for 24 hours to ensure cell adhesion. Then, cells were washed 3 times with PBS and culture medium was added according to experimental conditions. The last PBS wash was considered as time t=0.Cell proliferation was evaluated with the amount of DNA present in the petri dishes. Each experiment was performed in triplicate. The culture medium was changed daily. After 96 hours in culture, cells were lysed with Na-Citrate-SDS solution and incubated with Hoescht 33358. Samples were read at 365 nm with a spectrofluorometer. The results show a dose-response inhibition of endothelial cell proliferation with the pV compounds. The approximate IC₅₀ is 2 mM for bpV(phen) and 3.5 mM for bpV(pic).

FIG. 2: A and B show an anti-angiogenic response to bpV(phen), orthovanadate (Van) and protamine (Prot) using a vascularization test on the vitelline membrane of chick embryos. Each point represents a minimum of 15 embryos (15 to 28). In FIG. 2A), neovascularization was assessed using N/+scoring system; the proportion of embryos showing anti-angiogenesis increased with dosage, bpv(phen) being the most potent agent. In FIG. 2B, neovascularization was assessed using the 1-2-3 scoring system; the angiogenic score decreased with dosage, bpV(phen) being the most potent agent.

FIG. 3: Micrographs of the cell migration from aortic rings embedded in fibrin (×12). Compared to the sample control (A), cell migration was impaired in the presence of doses of bpV(phen) (1, 3.5 μM; B, C) with a strong inhibition at 3.5 μM (C). In the presence of bpV(pic) (3.5, 10, 25 μM; D, E, F), inhibition was seen at 10-25 μM. In the presence of orthovanadate, partial inhibition was reached at 25 μM (G) with no effect at 3.5 μM (H). The migration of well-defined microvessels in the presence of bpV(pic) is evident at 10 μM, while in the control sample very thin cell strands were frequently observed.

FIG. 4: Quantification of the cell migration from aortic rings in the presence of orthovanadate (plain bars); bpV(phen) (light gray bars); and bpV(pic) (dark gray bars). The control appears as an empty bar. Means and standard errors of the means are presented. The Student-Newman-Keuls method was used for statistical analysis (p value=0.001)

FIG. 5: bpV(phen) inhibits angiogenesis in vivo in the s.c. Matrigel assay. Graph showing the results of an experiment expressed as the number of cells that migrated in three microscopic fields for each s.c. Matrigel plug. Fields were equidistant from the edge of the plug. (1) Four mice that did not receive any treatment after the Matrigel plug implantation. (2) Four mice that received daily administration of 10 μg bpV(phen) during the 7 days post-implantation. (3) Four mice that received daily administration of 50 μg bpV(phen) during the 7 days post-implantation. (4) Four mice that received daily administration of 100 μg bpv(phen) during the 7 days post-implantation. (5) Four mice that received daily administration of PBS during the 7 days post-implantation.

FIG. 6: Neutrophils, eosinophils and MØ recruited in air pouch exudates of bpV(phen)-treated BALB/c mice in response to L. major promastigotes intra-pouch injection. Air pouch exudates were collected from animals injected with endotoxin-free PBS, LPS (20 μg/ml), L. major and bpV(phen) (500 nM i.p., 2 hour)+L. major, 6 hr post-inoculation. Each time point represents the mean±SD (n=4 mice) of 2 experiments similarly performed. Differences observed for specific leukocytes population recruited were significant (p<0.01, Student's t test) over their respective control.

FIG. 7: In vivo Leishmania-induced pro-inflammatory mediator generations in pV-treated mice. (A) Nitrate level determination in air pouch exudates collected 6 hr post-inoculation of endotoxin-free PBS, L. major and bpV(phen) followed by L. major has been performed. Values are the mean±SEM of 2 experiments performed independently. (B) Evaluation of pro-inflammatory cytokines (IL-6 and IL-1β) and chemokines (MCP-1 and MIP-2) secreted in air pouch exudates collected as described above were measured by ELISA as described in more detail below. Values are the mean±SEM of 2 experiments performed independently. Levels of pro-inflammatory molecules measured in exudate supernatants were significantly augmented (p<0.01, Student's t test) in response to L. major infection and significantly further increased by bpV(phen) treatment over their respective control. IL-6, IL-1β, MCP-1 and MIP-2 secretion were respectively 12.8-, 2-, 8- and 6.8-time more secreted in L. major -injected bpV(phen)-treated animals compared to L. major alone. PBS control was equal to background.

FIG. 8: In vivo bpV(phen)-induced cytokine gene expression in spleen of BALB/c mice. Expression of Th1 (IFN-(, IL-2, IL-12) and Th2 (IL-4 and IL-10) cytokines were monitored in spleen of BALB/c mice in response to bpv(phen) over a 24-hr period. Expression of cytokine genes has been monitored using a multi-probe RNase protection assay as described in more detail below. Results obtained are representative of 3 experiments performed independently.

FIG. 9: Effect of bpV(phen) on chemokine mRNA expression in murine B10R macrophages. (A) Cells were incubated for 2 h with or without various concentrations of bpV(phen) (1-50 μM). Total RNA was isolated and analyzed by RNase protection. (B) Kinetic expression of chemokine genes in murine BIOR macrophages. Cells were incubated for increasing period of time (1-4 h) with or without bpV(phen) at 10 μM. Following extraction of total RNA, RNase protection assay was performed. Free RNA probe is shown in the far left lane. The fold increase was calculated from the densitometry of the autoradiogam.

FIG. 10: Use of bpV(phen) as an adjuvant in Leishmania vaccination trial. BALB/c mice were inoculated with soluble Leishmania major antigen (SLA; 100 μg) with or without daily injection of bpV(phen) (500 μM) for 5 days. Two weeks later animals received a second set of treatment as above. Control animals received either PBS or bpV(phen) alone without any SLA. At 4 weeks post-vaccination, animals were challenged with 5 million Leishmania major stationary phase promastigotes injected in the right hind footpad. Progression of infection was followed by measuring footpad thickness with a caliper on a weekly basis. Differences measured for reduced footpad thickness were significant (p<0.01, Student's t test, n=5-10 animals per group) over their respective controls.

FIG. 11: Illustration of the antitumor activity of bpV(phen) in vitro using PC3 prostate cancer cells. Collagen gels containing the PC3 cells were prepared according to the method of Esdale and Bard (1972). Briefly, stock collagen solution (3.5 mg/ml in acetic acid 0.02N; Rat tail) was added to a mixture composed of culture medium (5×), fetal bovine serum (FBS), bicarbonate (0.26M), and was neutralized with 0.1N NaOH. A cell suspension (1.42×10⁶ cells/ml) was mixed into the collagen-medium mixture to obtain a final concentration of 2.5×10⁵ cells/ml.

A DMEM medium, having a normal concentration of glucose and without phenol red, was used. After gelification (within 1 h) of the collagen mixture containing the cells, the gels were removed from their culture wells (mould) and interwoven into a receptor hole prepared in a fibrin gel, as previously described (103). The fibrin gel was made from a 0.3% fibrinogen solution in Hank's balanced salt solution. Fibrin was allowed to polymerize with thrombin (stock solution at 1.75 mg/ml) at a ratio of 1:003 v/v fibrin to thrombin. The collagen-fibrin complexes were then covered with serum-supplemented medium according to the cell types. An inhibitor of plasminogen activator (Trasylol, Parke Davies) was added into the medium at 10 μl/ml (100U/ml).

Cell behavior was periodically monitored over 15 days of culture. In this model, collagen is believed to mimic the tumor stroma, and fibrin is well recognized as the primary matrix for cancer cell expansion and migration (104). bpV(phen) was used at 2, 5 and 10 μM; the compound was renewed daily over a 2 week period. Phase contrast microscopic observations and micrographs were taken after 15 days, and samples were prepared for histological sections. Histological sections were stained with periodic acid Shiff stain to enhance the matrix contrast.

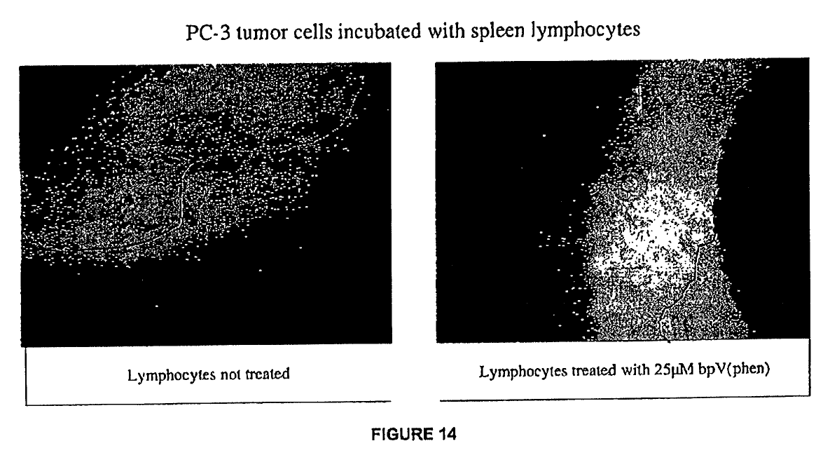

FIG. 12: Illustration of the antitumor activity of bpV(phen) in vitro using ZR-75 breast cancer cells. Experiments were done as described for the FIG. 11 except that DMEM medium having a high glucose concentration and without phenol red was used. 10% FBS was used instead of 5% FBS. The media was supplemented with 10⁻⁹ M estradiol (final concentration).

FIG. 13: FIGS. 13A and 13B illustrates the antitumor activity of bpv(phen) in vivo.

FIG. 14: In vitro antitumor activity of lymphocytes pretreated with bpV(phen). PC-3 cancer cells embedded in a collagen gel, grew as a “primary tumor”. Some cells migrated from the primary tumor toward the fibrin gel, forming front edges that can be quantified. Small clumps of cells progressively appear in the fibrin gel and we assume that these cell extension are representative of the invasive potential of the cancer cells. In the control panel (left), PC-3 cells migrated slightly from the primary tumor and formed extensive secondary tumors in the fibrin gel. In the presence of bpV(phen)-treated lymphocytes no secondary tumor was observed and there was no migration front. In addition, the primary tumors appeared less dense than in the control.

### DEFINITIONS

In order to provide a clear and consistent understanding of terms used in the present description, a number of definitions are provided hereinbelow.

Unless defined otherwise, the scientific and technological terms and nomenclature used herein have the same meaning as commonly understood by a person of ordinary skill to which this invention pertains.

For the purposes of the present application, the term “animal” is meant to signify human beings, primates, domestic animals (such as horses, cows, pigs, goats, sheep, cats, dogs, guinea pigs, mice, birds, fish etc.).

From the specification and appended claims, the term “therapeutic agent” should be taken in a broad sense so as to also include a combination of at least two such therapeutic agents.

For administration to humans, the prescribing medical professional will ultimately determine the appropriate form and dosage for a given patient, and this can be expected to vary according to the chosen therapeutic regimen, the response and condition of the patient as well as the severity of the disease.

Compositions within the scope of the present invention should contain the active agent (e.g. compound) in an amount effective to achieve the desired therapeutic effect while avoiding adverse side effects. Typically, the compounds of the present invention can be administered to mammals (e.g. humans) in doses ranging from 0.001 to 50 mg per kg of body weight per day of the mammal which is treated. Pharmaceutically acceptable preparations and salts of the active agent are within the scope of the present invention and are well known in the art (Remington's Pharmaceutical Science, 16th Ed., Mack Ed.). The dosage will be adapted by the clinician in accordance with conventional factors such as the extent of the disease and different parameters from the patient. Typically, 0.001 to 50 mg/kg/day will be administered to the mammal.

A) Peroxovanadium Compounds and Angiogenesis

1. Peroxovanadium Compounds Inhibit the Proliferation of Endothelial Cells and the Formation of Tubular Structures

Human umbilical vein endothelial cells (HUVECs) were extracted with collagenase-controlled digestion, as previously described (105). Pure HUVECs were used before the fourth passage (trypsin-EDTA at each passage). The cells were analyzed for their capacity to incorporate di-acetyl LDL and to be labelled with factor VIII-related antigen.

Endothelial cells were plated at a density of 2500 cells/cm² in a sterile plate coated with gelatin. Cells were cultured in complete medium (M199: heparin (90 μg/ml) or L-glutamine (2 mM), bicarbonate, FBS (20%) and ECGS (100 μg/ml)) for 24 hours to ensure cell adhesion. Then, cells were washed 3 times with PBS and culture medium was added according to experimental conditions. The last PBS wash was considered as time t=0.

Cell proliferation was evaluated with the amount of DNA present in the petri dishes. Each experiment was performed in triplicate. The culture medium was changed daily. After 96 hours in culture, cells were lysed with Na-Citrate-SDS solution and incubated with Hoescht 33358. Samples were read at 365 nm with a spectrofluorometer.

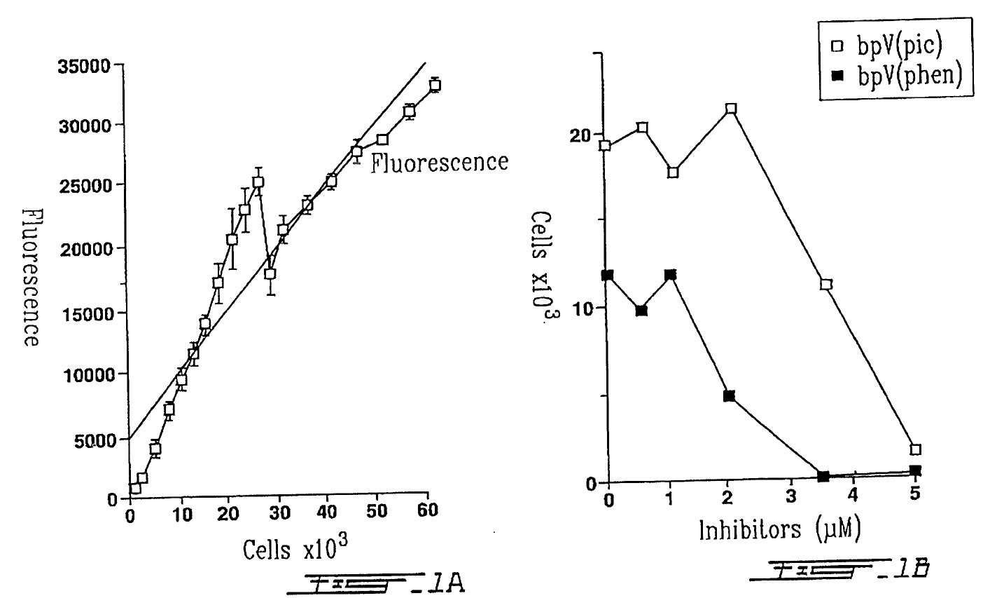

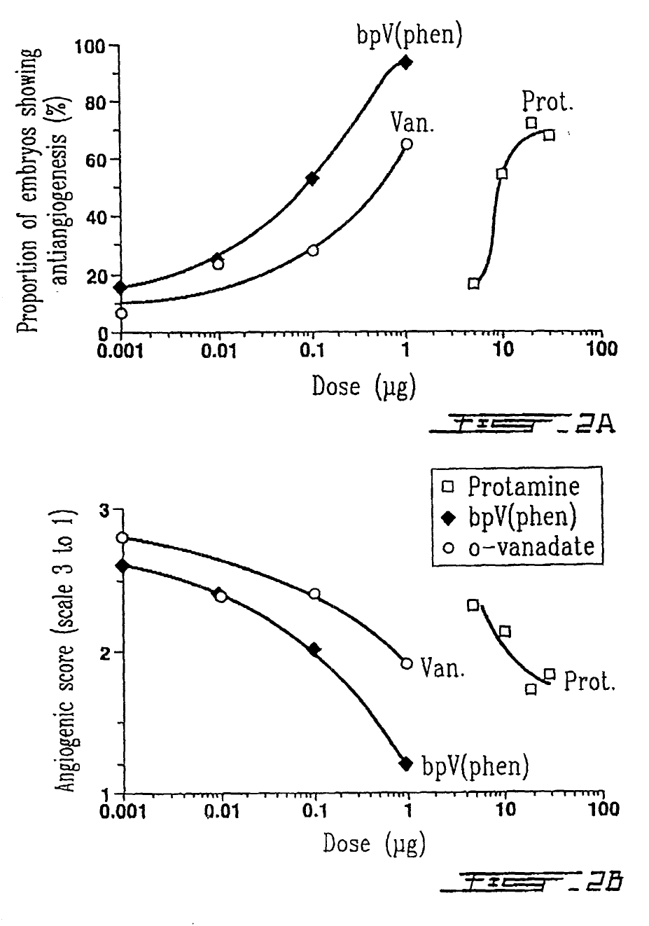

The results show a dose-response inhibition of endothelial cell proliferation with the pV compounds (FIG. 1). The approximate IC₅₀ is 2 μM for bpV(phen) and 3.5 μM for bpV(pic).

Alternatively, HUVECs cultured in a fibrin matrix can form 3-dimensional tubular-like structures in the presence of serum (103). This assay was performed in the presence of either bpV(phen), bpv(pic) or vanadate to assess the influence of each one of these compounds on HUVEC differentiation and organization (Table 1). HUVECs were seeded on the bottom of gelatin-coated wells at high density to provide a confluent monolayer at 48 hours.

Then, 50 000 HUVECs/ml were embedded in fibrinogen solution prior to polymerization. The fibrin matrix was covered with culture medium containing the molecule to be tested, while culture medium without any of these molecules served as controls. Cell behavior was observed periodically by phase contrast microscopy. After 21 days in culture in the presence of 1 μM bpV(phen), cord-like structures (or cords), tube-like structures (or tubes) and stellate structures (or Stell.struct.) were observed. At higher doses (2 and 3.5 μM), fragmented cord-like structures were apparent. In the presence of bpV(pic), cords and tubes were observed at 1, 2 and 3.5 μM. In the presence of orthovanadate, cords were still apparent at 10 μM, and at 25 μM dead cells were sparsely distributed. These results suggest that pV compounds interfere with endothelial cell organization and terminal differentiation. Furthermore, the nature of the ancillary ligand is important since bpV(phen) is more potent in inhibiting tube formation than bpV(pic).

The results show that vanadate has an anti-angiogenic effect inasmuch as the stellate structures are affected (organization and terminal differentiation). Vanadate is at least 3 and 5 times less potent than bpV(pic) and bpV(phen), respectively. The anti-angiogenic effect observed with vanadate is in contradiction to the teachings of Montesano (47).

Indeed, it was observed that at low doses, all the tested vanadium compounds seemed to be pro-angiogenic, while at higher doses (i.e., at almost double the dose), the same compounds were clearly anti-angiogenic. The type of radical L, L′ greatly affects the potency of the vanadium compounds; for example, phenanthroline was found to be twice as potent as picolinic acid.

TABLE I
Effects of bpV(phen), bpV(pic) and Vanadate on HUVEC
Differentiation and Organization

| | Doses | | | Stell. |
|---|---|---|---|---|
| | (μM) | cords | tubes | struct. |
| bpV | 0 | +++ | +++ | +++ |
| (phen) | | | | |
| | 1 | +++ | +++ | +++ |
| | 2 | ++ | + | − |
| | 3.5 | − | − | − |
| bpV (pic) | 0 | +++ | +++ | +++ |
| | 1 | +++ | +++ | +++ |
| | 2 | +++ | +++ | +++ |
| | 3.5 | ++ | + | − |
| van | 0 | +++ | +++ | +++ |
| | 2.5 | +++ | +++ | +++ |
| | 10 | +++ | +++ | − |
| | 25 | † | † | † |

† Dead cells

2-Rat Aortic Ring Assay

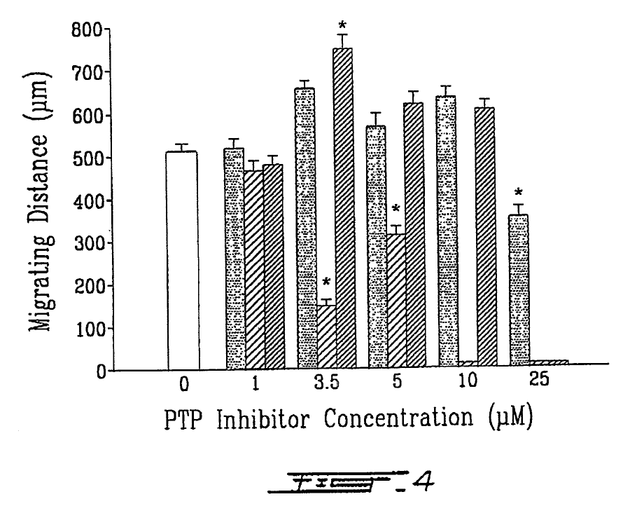

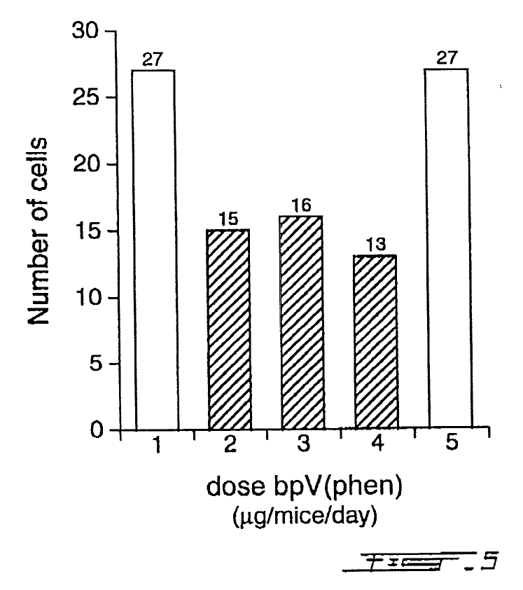

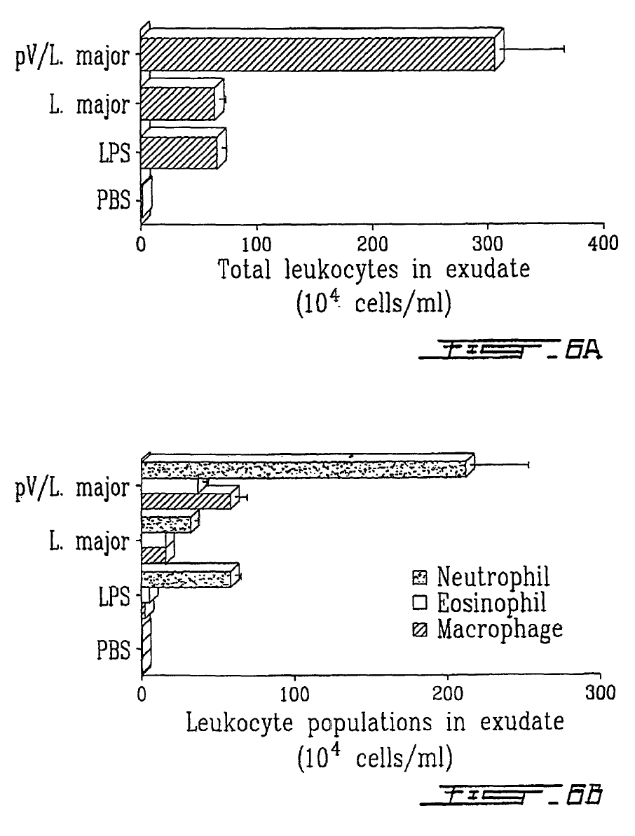

Rat aorta rings were embedded in fibrin matrix as previously described (107). Thoracic aortas excised from adult rats were rinsed in Hank's balanced salt solution (HBSS), and cleansed of periadventitial. One-mm long aortic rings were sectioned and rinsed in culture medium. Each of the aortic rings were then embedded in fibrin gel matrix. Migration of microvessels in fibrin gel was quantified by measuring the distance from the external surface of rat aortic rings towards the migrating vessels. Measurements were performed at day 15 by digitizing morphometry with a NIH image analysis system (FIGS. 3 and 4).

3-Peroxovanadium Compounds Inhibit Neovascularization ex ovo

a. Definition of the Test-System:

The normal development of a chick embryo involves the formation of an external vascular system which is located in the vitelline membrane and which carries nutrients from the vitellus (yolk of the egg) to the developing embryo. When placed onto the vitelline membrane, anti-angiogenic substances can inhibit blood vessel development in the vitelline membrane. To facilitate access to the vitelline membrane, chick embryos were transferred to a sterile culture box (Petri dish) and placed in a humidity- and temperature-controlled incubator. Embryos could then develop in this ex ovo condition for several days.

An aliquot of the tested compound was mixed with a methylcellulose solution and allowed to air-dry into thin discs. Methylcellulose forms an inert matrix from which the tested compound can diffuse slowly. Methylcellulose discs containing the tested compound were placed on the external border of the vascular perimeter of the vitelline membrane where the angiogenic process was still active.

The effects of the discs containing the tested compound on proximal vascular development were, in all cases, compared with those of discs containing control buffer. The discs were placed on the embryos' vitelline membrane on Day 0 or Day 1 of the ex ovo growth process; at this point, only beginnings of the main blood vessels are invading the vitellus. The embryos were then put in culture conditions until vascularization was assessed (approximately 24 h). Control buffer- and compound-containing discs were in all cases added simultaneously on the vitelline membrane of the same embryo. Both discs were arranged in a symmetrical fashion with respect to the cephalo-caudal axis of the embryo in order to minimize inter-individual variations when comparing the tested compounds with controls.

b. Anti-Angiogenic Activity:

Embryonic vascularization tests (EVTs) were performed using different concentrations of protamine (5 to 20 μg) as a positive control or a tested compound (0.001 to 10 μg). After one day in culture, the level of vascularization in the area covered by the discs was graded by a pair of scientists in the usual blind fashion. To facilitate the location of the discs, black O-rings were placed around them just after their placement on the vitelline membrane. The evaluation scale for the EVTs was based on two different scoring systems.

Assessment of Blood Vessel Formation:

Blood vessel formation was assessed in a blind fashion. The areas of the vitelline membranes that lay beneath the methylcellulose discs were scored for the degree of vascularization, using two scoring systems (“N/+” or “1-2-3”). The following selection criteria were used:

N/+System:

A score of “N” for “Normal” was attributed when all the following conditions were met:

Blood vessels in the evaluated area grew along their path with no abnormal deviation. Collateral branching density was normal and the growth path of the lateral branches was also normal.

A score of “+” was attributed when at least one of the following conditions was met:

Major blood vessels grew across the evaluated area but their paths were clearly affected (winding).

Major blood vessels grew across the evaluated area but collateral branching density was clearly diminished.

Major blood vessels penetrated the evaluated area but their growth path rapidly deviated.

A kink was observed in the blood vessel. Major blood vessels penetrated the evaluated area but were stunted. No growth was observed beyond that point.

A drastic deviation in the growth path occurred when major blood vessels reached the proximal ridge of the disc.

1-2-3 Grading Scale System:

A score of 3 was attributed when the following conditions were met:

Blood vessels, present in the evaluated area, grew along their paths with no abnormal deviation. Collateral branching density was normal and the growth paths of the lateral branches were also normal.

A score of 2 was attributed when at least one of the following conditions was met:

Major blood vessels grew across the evaluated area but their paths were clearly affected (winding).

Major blood vessels grew across the evaluated area but collateral branching density was clearly diminished.

A score of 1 was attributed when at least one of the following conditions was met:

Major blood vessels penetrated the evaluated area but their growth paths were rapidly deviated.

A kink was observed in the blood vessels.

Major blood vessels penetrated the evaluated area but were stunted. No growth was observed beyond that point.

A drastic deviation in the growth paths occurred when major blood vessels reached the proximal ridge of the disc.

A score of 3 meant that blood vessel development was normal whereas a score of 1 indicated the greatest degree of angiostatic activity.

TABLE II
Data on the Angiostatic Action of Bpv(phen), Orthovanadate, and Protamine on the
Neovascularisation of the Vitelline Membrane of Chick Embryos.

| | Assay 1 | | | Assay 2 | | | Assay 3 | | | Assay 4 | | | Assay 5 | | |
|---|---|---|---|---|---|---|---|---|---|---|---|---|---|---|---|
| Substance(: | Anti-angio.activity | | | Anti-angio.activity | | | Anti-angio.activity | | | Anti-angio.activity | | | Anti-angio.activity | | |
| g) | N¹ | % | 1 to 3 | N¹ | % | 1 to 3 | N¹ | % | 1 to 3 | N¹ | % | 1 to 3 | N¹ | % | 1 to 3 |
| Protamine-5 | 11 | 23 | 2.3 | — | — | — | — | — | — | 11 | 9 | 2.3 | 22 | 16 | 2.3 |
| Protamine-10 | — | — | — | — | — | — | — | — | — | 12 | 54 | 2.1 | 12 | 54 | 2.1 |
| Protamine-20 | 12 | 46 | 2.0 | 15 | 80 | 1.5 | 15 | 79 | 1.5 | 12 | 80 | 1.8 | 54 | 71 | 1.7 |
| Protamine 30 | — | — | — | — | — | — | — | — | — | 12 | 67 | 1.8 | 12 | 67 | 1.8 |
| bpV(phen)-0.001 | 13 | 12 | 2.7 | 15 | 20 | 2.4 | — | — | — | — | — | — | 28 | 16 | 2.6 |
| bpV(phen)-0.01 | 13 | 31 | 2.3 | 13 | 19 | 2.5 | — | — | — | — | — | — | 26 | 25 | 2.4 |
| bpV(phen)-0.1 | 14 | 61 | 1.8 | 14 | 45 | 2.2 | — | — | — | — | — | — | 28 | 53 | 2.0 |
| bpV(phen)-1 | 11 | 86 | 1.6 | 12 | 100 | 1.1 | — | — | — | 10 | 96 | 1.0 | 23 | 94 | 1.2 |
| o-Van-0.001 | — | — | — | — | — | — | 15 | 7 | 2.8 | — | — | — | 15 | 7 | 2.8 |
| o-Van-0.01 | — | — | — | — | — | — | 15 | 24 | 2.4 | — | — | — | 15 | 24 | 2.4 |
| o-Van-0.1 | — | — | — | — | — | — | 16 | 28 | 2.4 | — | — | — | 16 | 28 | 2.4 |
| o-Van-1 | — | — | — | — | — | — | 15 | 70 | 2.0 | 7 | 57 | 1.7 | 22 | 64 | 1.9 |
| Control | | | | 69 | 17 | 2.6 | 79 | 9 | 2.5 | 67 | 8 | 2.8 | 21 | 11 | 2.6 |
| | | | | | | | | | | | | | 5 | | |

N¹ = number of tested embryos;
% = proportion of embryos showing an anti-angiogenic response
1-3 = Semi-quantitative scale of angiogenesis (1: no angiogenesis; 3: normal angiogenesis)

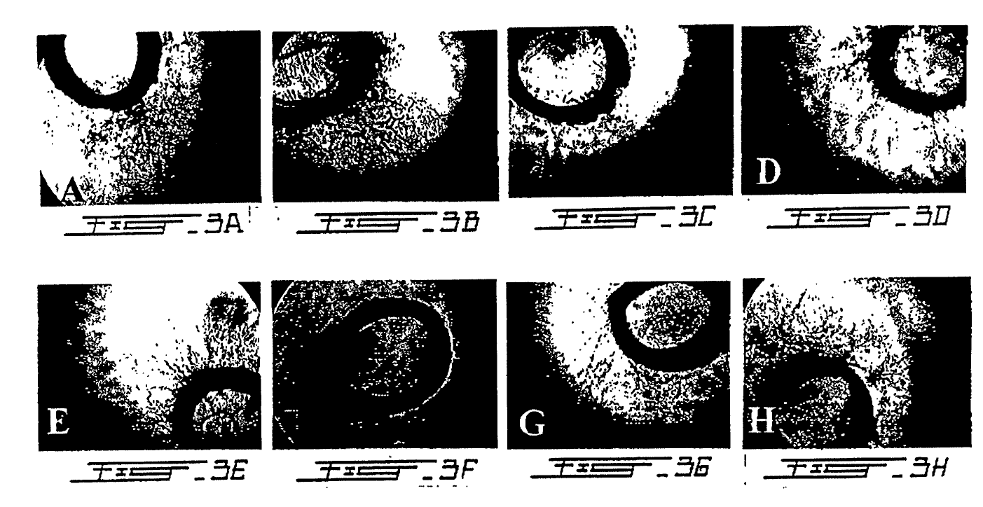

A dose-response inhibition was obtained with protamine, bpV(phen), and orthovanadate (FIG. 2). The approximate IC₅₀ is 0.1 μg for bpV(phen), 0.6 μg for orthovanadate, and 11 μg for protamine (regardless of the scoring system). A summary of the experimental data collected is shown in Table II.

The results show that bpV(phen) is a potent inhibitor of the angiogenic process. Prior art on the subject (vanadate) taught the opposite, namely that vanadium compounds promote angiogenesis.

4-In vivo Matrigel Plug Assay

This assay was performed as previously described (107). Briefly, Matrigel (liquid at 4° C.) was mixed with 200 ng/ml ECGS and injected s.c. into four C57B1/6N female mice (1 ml/mice). After injection, the Matrigel polymerized to form a plug. After 7 days, the animals were sacrified, and the Matrigel plugs were removed and fixed in 10% neutral-buffered formalin solution (Sigma Chemical Co.) and embedded in paraffin. Histological sections were stained with Masson's trichrome and angiogenesis was scored by counting the number of cells that migrated in three microscopic fields for each s.c. Matrigel plug.

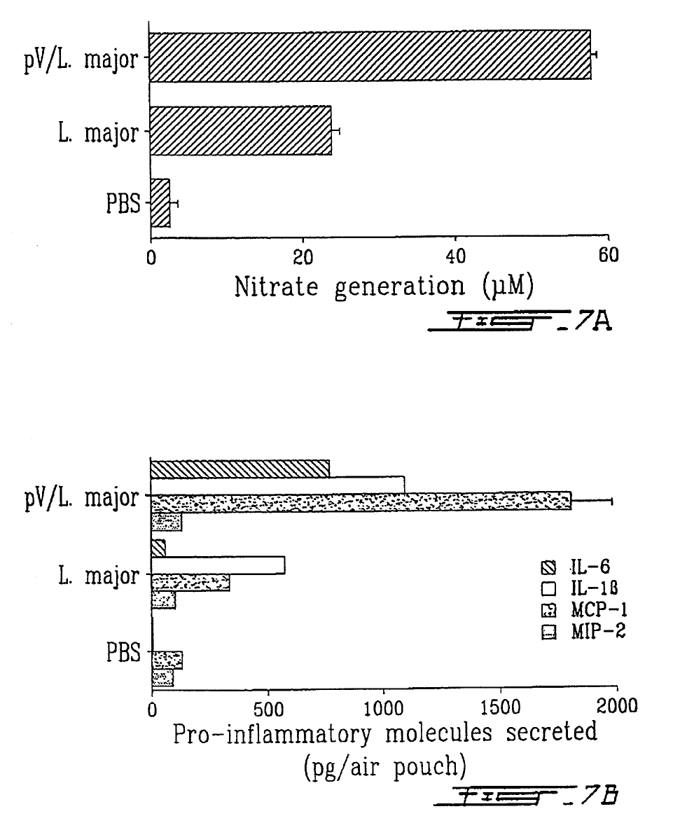

Experiments were performed to determine whether bpV(phen) inhibits vessel formation in vivo. Animals were divided into five groups (4 mice/group): two control groups that received no treatment or daily i.p. PBS injection, respectively, and three groups that received daily administration of various doses (10, 50 and 100 μg) of bpV(phen), from day 1 to day 7 (intraperitoneal daily administration of 100 μl solution). The results show that bpV(phen) is a potent angiogenesis inhibitor in vivo (FIG. 5). Daily administration of 10 μg/mice or higher doses decreased by half the number of endothelial cells having invaded Matrigel plugs.

The angiogenic potential of pV compounds was tested using in vitro, ex ovo, and in vitro systems of analysis. The results show that in contrast to what might have been expected from the teaching of the prior art, pV compounds are potent inhibitors of the angiogenic process (see below), and this includes high doses of vanadate.

B) Peroxovanadium Compounds and the Production of Endothelins

The data presented in Table III show the effect of bpV(phen) on the plasmatic levels of ETs. In this study, rats were injected intraperitoneally with bpv(phen) (0.5 mg/100 g b.w.) 16 hours before the administration of either insulin or vehicle. Two minutes following insulin administration (1.5 μg/100 g b.w, or vehicle), the plasma levels of ETs were determined. Insulin administration induced a strong increase of seric ET concentration (54). This increase was completely abolished by bpV(phen). In addition, bpV(phen) decreased the insulin-stimulated levels of plasmatic ETs below control levels. These results suggest that bpV(phen) inhibits the insulin-induced release of ETs that subsequently can lead to diabetic complications like vasculopathy and nephropathy.

TABLE III
Effect of bpV (phen) on Endothelin Plasma Levels
Plasma endothelins (pg/ml)

| | |
|---|---|
| Control | 113.41 ± 10.91 |
| Insulin | 253.10 |
| Insulin, bpV(phen) | 77.68 ± 3.08 |

ETs (ET-1, ET-2, ET-3) were measured by RIA (Amersham kit RPA 555) after lyophilization and extraction on C2 columns (500 mg). Results are mean ±SEM, p<0.0209, control (n=4) vs Insulin, pV (n=3).

C) Peroxovanadium Compounds and Restenosis

The data presented in Table IV show the effect of the pV compound bpV(bipy) on restenosis. Male Sprague-Dawley rats (325-350 g) were anaesthetized with Rogarsetic, a mixture of ketamine hydrochloride and xylazine hydrochloride. The animals were divided into two groups of 14-31 rats that underwent balloon angioplasty of the left carotid artery, with or without treatment (108). The treated group received bpV (bipy) (10 mg/kg/i.p. b.i.d.) for 14 days, starting on the day of the angioplasty. Every animal in each group underwent balloon angioplasty of the left external carotid whereas the contra-lateral carotid served as a control for each animal. The left carotid artery balloon angioplasty was performed under aseptic conditions. The left external carotid was isolated and an arteriotomy was performed. A 2F Fogarty arterial embolectomy catheter was then inserted in the left common carotid and positioned near the aortic arch. The balloon was then inflated and retracted with a twisting motion to its insertion point. This procedure was repeated twice and the external carotid was ligated. There were no differences in the lumina (L), intima (I), media (M), the ratio of I/M and calculated value of (M−L/P) as a measure of neointimal proliferation (NIP) between normal, non-ballooned carotid artery of treated and non-treated rats, serving as controls (data not shown). Treatment caused a 32% decreased in NIP and the I/M ratio, a 21% reduction in the thickness of the I and a 28% increase in the opening of the L. Thus, treatment with bpV (bipy) revealed a significant reduction in the degree of post-angioplastic vascular remodeling of the carotid artery in the rat model.

The above results illustrate the antiangiogenic and anti-restenosis potential of vanadium compounds. The administration of pV compounds could be used to control the progression of several angiogeno-dependent conditions as well as post-angioplastic vascular remodeling. Since vanadates are anti-tumor compounds, the present pV compounds should function as anti-tumor therapeutic agents, and part of this activity should be due to an anti-angiogenic effect. Pharmaceutical compositions would comprise potent pV compounds capable of achieving an extracellular concentration of about 0.1 to 100 μM, preferably 2-40 1 μM. In rats, a dose of 20 μmoles per kilogram was successful in reversing the endothelin increase after insulin administration. Moreover, pV compounds, which as previously stated are more potent and provoke less side effects than the vanadium “oxo” compounds, can be administered to achieve the desired dosage. For example, see in vivo effects on blood glucose levels have been reported (1, 3).

TABLE IV
Effect of bpV (bipy) on Restenosis

| | NIP | I | | | L |
|---|---|---|---|---|---|
| | mm | mm² | M | I/M | mm² |
| Control | 0.064 | 0.138 | 0.090 | 1.546 | 0.090 |
| bpV(bipy) | 0.045 | 0.112 | 0.104 | 1.086 | 0.112 |
| p < x | 0.002 | 0.04 | 0.005 | 0.003 | 0.003 |

Abbreviations:
I, intima;
M, media;
NIP, neointimal proliferation;
L, lumina.

Abbreviations: I, intima; M, media; NIP, neointimal proliferation; L, lumina.

D) Peroxovanadium Compounds and the Immune Response

The present invention provides pV compounds as novel immunomodulator and adjuvant molecules. The pV compound bpV(phen) can be used as an immunomodulator in vitro (macrophage; chemokine genes expression) and in vivo (murine model; cytokine genes expression, cytokine and chemokine generations and cellular recruitment in response to various stimulation), and as an adjuvant to maximize the protective effect of known Leishmania antigen based vaccine. The above bpv(phen) compound is a potent immunomodulator based on its capacity to induce cytokine (e.g. IL-12, IFN(, IL-1β) and chemokine (e.g. RANTES, MIP-1a,β, MIP-2, IP-10, MCP-1) gene expression to enhance cellular recruitment in response to an agonist and consequently act as an adjuvant in the context of vaccination.

1-bpV(phen) Treatment Enhance Pro-Inflammatory Molecules Secretion and Polymorphonuclear Recruitment in Response to Leishmania Infection

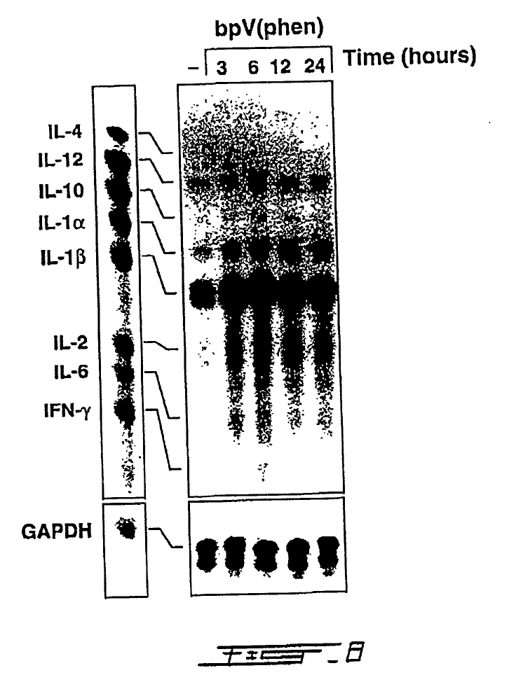

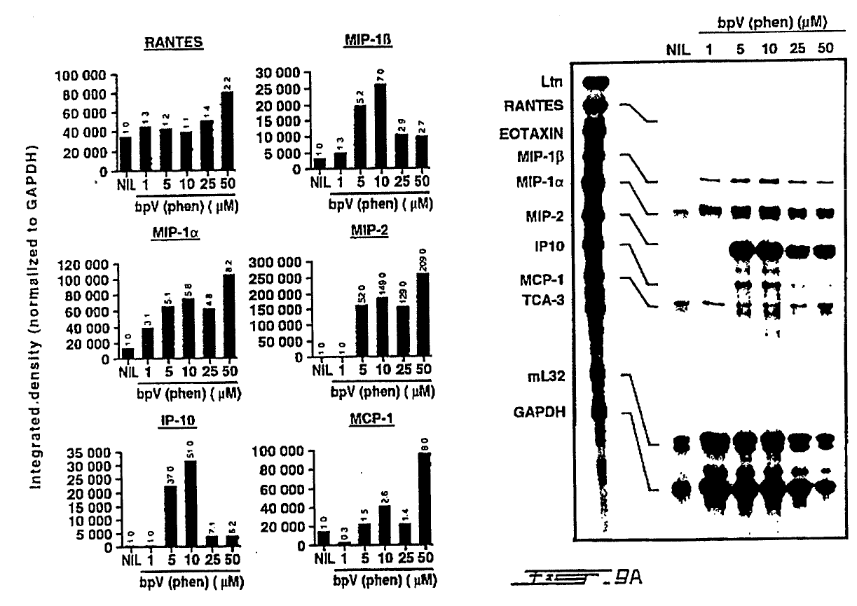

With the use of a murine air pouch model, we investigated early inflammatory events that occur following L. major skin injection in bpV(phen)-treated animals (FIG. 6). As shown in FIG. 6A, both L. major and LPS (positive control) could induce significant leukocyte recruitment in air pouch exudates as measured 6 hours post-injection. Of interest, bpV(phen)-treated BALB/c mice, in response to L. major promastigotes (10⁷ parasites/ml) intra-pouch injection, manifested a five times greater recruitment of cells within the same time period. In addition, differential counts performed on Diff-Quick-stained cytospin preparations from these cells (FIG. 6B) revealed that ˜70% of recruited leukocytes were neutrophils (18% eosinophils and 12% macrophages), whereas in untreated animals receiving L. major promastigotes ˜48% of recruited cells were neutrophils with the rest of the cell population consisting of eosinophils (26%) and macrophages (26%). This data revealed that bpV(phen) is a good modulator of the early inflammatory response and could have a major impact on the progression of L. major infection since the dramatic increase in inflammatory cell recruitment consisted mainly of neutrophils, already recognized for their importance to restrain Leishmania pathogenesis (109).

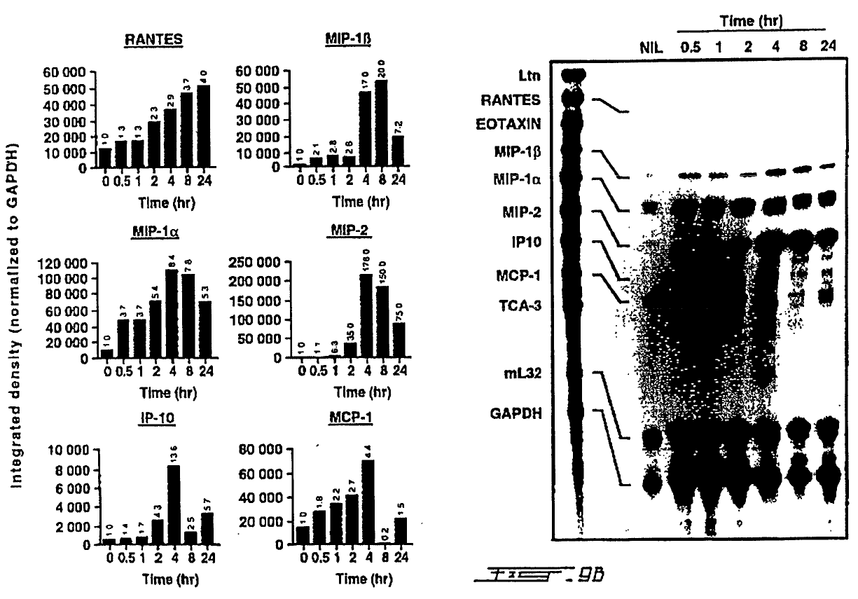

In parallel to this experiment, in vivo generation of Leishmania-induced NO and pro-inflammatory mediators in bpV(phen)-treated mice were measured in air pouch exudates (FIG. 7).

The present set of data reinforce the idea that bpv(phen) is an excellent NO modulator as revealed by its enhanced generation in response to L. major infection (FIG. 7A). This elevated NO generation not only further increased the microbicidal activity observed in bpV(phen)-treated animals, but also partly explained the strong inflammatory response observed in bpV(phen)-treated animals since NO has been recognized as an important mediator of inflammation and regulator of neutrophil migration (110, 111). In addition, bpV(phen)-treatment has shown elevation of some pro-inflammatory cytokines (IL-6 and IL-1β) and chemokines (MCP-1 and MIP-2) measured in air pouch exudates of animals inoculated with L. major promastigotes (FIG. 7B). MIP-2 chemokine is recognized as a specific chemoattractant for neutrophils (112) whereas MCP-1 has been shown to be a powerful monocyte/macrophage recruiter to sites of inflammation. Additionally, the pro-inflammatory cytokines IL-1β and IL-6 are well known chemokine modulators playing a pivotal role in the development of inflammation (113) including the regulation of MCP-1 and MIP-2 expression (114).

2-Up-Regulation of IL-12, IL-2 and IFNγ mRNA Expression in Spleen of bpV(phen)-Treated Animals

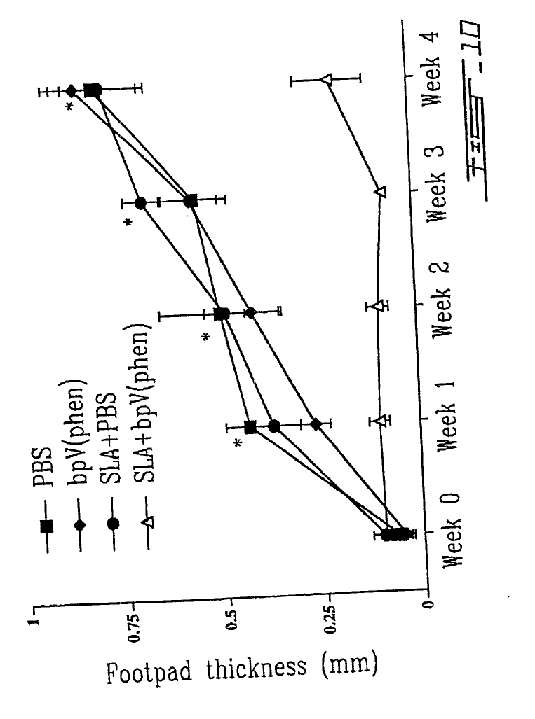

As shown in FIG. 8, in vivo bpV(phen)-induced cytokine gene expression-monitored with the use of a multi-probe RNAse protection assay-in spleen of naive mice was significantly up-regulated for up to 24 hr (i.e. IL-12, IL-10, IL-2 and IFN( ) in comparison to animals injected with PBS. Our observation provides evidence that the PTP inhibitor bpV(phen) is a powerful immunomodulator favoring the activation of protective Th1 type cytokines recognized to modulate NO generation in vivo resulting in control over cutaneous leishmaniasis (115, 116). Additionally, whereas little is known concerning the role of PTPs on the regulation of cytokines, the present observation revealed that different signaling events being or not under the control of PTPs conduct to cytokine genes regulation. Overall, these last experiments clearly demonstrated the capacity of bpV(phen) to up-regulate several protective inflammatory and immunological functions of the host in response to a pathogen.

3-Effect of bpV(phen) on Chemokine Gene Expression

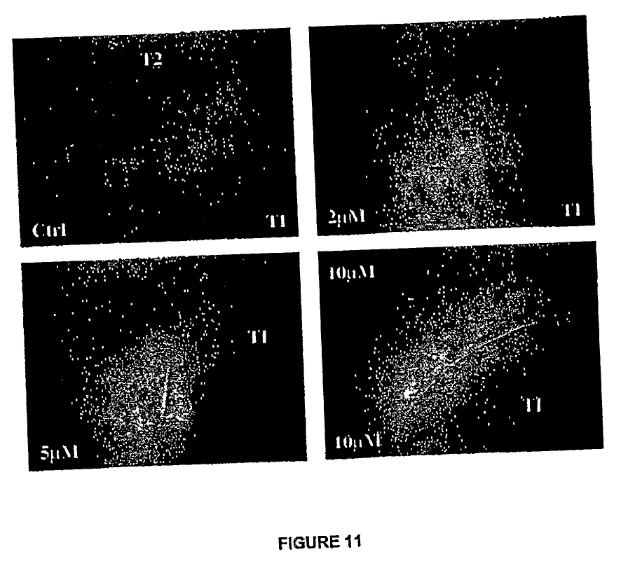

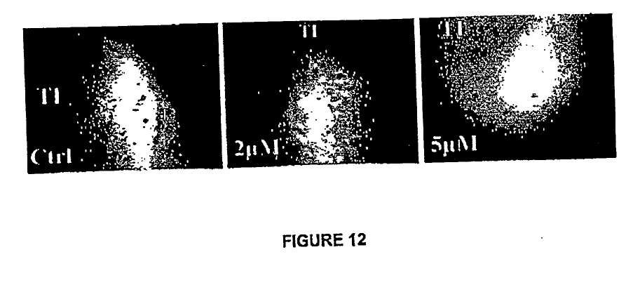

Various concentrations of bpv(phen) were added to B10R macrophages for4 h. mRNA expression was measured by RNase protection assays. RANTES, MIP-1a/β MIP-2, IP-10 and MCP-1 chemokine mRNA levels increased in response to bpv(phen) (FIG. 9A). MCP-1, MIP-2, MIP-1β and RANTES were induced by the addition of bpv(phen) in a dose-dependent manner whereas IP-10 and MIP-1a were expressed at markedly lower concentrations (10 μM). Thus, bpV(phen) used at 10 μM seems to be the most effective dose for the induction of chemokine gene expression. In addition, we noted that their expression was were differently modulated by bpV(phen) treatment, suggesting that different type of PTPs must play a specific role in the regulation of various chemokines.

To determine the kinetics of chemokine expression following pV compound treatment, B10R macrophages were incubated with 10 ?M bpV(phen) for increasing time intervals. Total RNA was extracted from macrophages at various time-points and subjected to RNase protection assay. There was a transient induction of MIP-1a, MIP-2, IP-10, and MCP-1 chemokines mRNA, reaching maximum levels at 4 h and rapidly declining afterward (FIG. 9B), whereas for MIP-1β, the optimal expression was achieved at 8 h post-treatment. However, the expression of RANTES mRNA increased in a time-dependent manner over 24 h (FIG. 9B). Thus, conditions for the induction of chemokine expression in response to bpV(phen) seems to vary with the different chemokines.

These results clearly showed that bpV(phen) had a stronger effect on some chemokines than on others. Evaluation of mRNA expression by densitometry analysis (data not shown) demonstrated a greater induction of MIP-2>MIP-1a>MCP-1 in presence of bpV(phen) in comparison to MIP-1β>RANTES>IP-10.

We report that modulation of host PTPs by bpV(phen) was effective in inducing RANTES, MIP-1a, MIP-1β MIP-2, IP-10 and MCP-1 chemokine gene expression in B10R murine macrophages. However, its action varied according to each chemokine, demonstrating a more potent effect on specific chemokines. Indeed, RNase protection assays have shown a greater induction of MIP-2>MIP-1a>MCP-1 by adding bpV(phen) compared to MIP-1β>RANTES>IP-10. Thus, our results designate PTPs as important negative regulators of the signaling process implicated in the chemokine production when they are activated. This is consistent with several studies where PTPs negatively regulate many cellular signaling such as B-cell receptor (BCR) (117) and erythropoietin receptor (118).

4-Use of bpV(phen) as Adjuvant in the Context of Leishmania Vaccine Trial

The adjuvant potential of bpV(phen) was tested in vivo in the context of Leishmania infection and resulted in complete protection against infectious challenge.

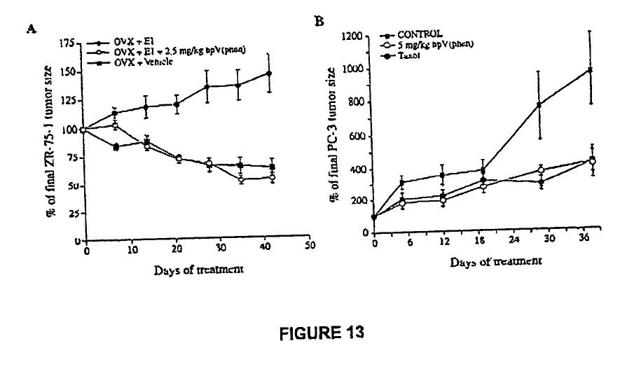

In the past, use of total and soluble Leishmania antigen (SLA) for vaccination has been reported and permitted some levels of protection against infectious challenges (85). Using a murine model, we have tested whether bpV(phen)-modulated cytokine and chemokine generation (as reported above) in combination with SLA administration could lead to protection against Leishmania infection. Mice have received PBS and bpv(phen) as controls, and SLA (100 μg) with or without daily bpV(phen) injection (500 nM, 5 days). All injections were done intra-peritoneally. Two weeks later, all groups were inoculated similarly with their respective treatment. Finally, 4 weeks post-vaccination, all animals were challenged with infectious Leishmania (5×10 L. major intradermally injected in the right hind footpad) and progression of infection followed over a period of 4 weeks post-infection (FIG. 10). Protection mediated by SLA+bpV(phen) has been successful since no significant footpad inflammation and skin lesion development was observed in comparison to all the other experimental groups.

E) Peroxovanadium Compounds and the Inhibition of Tumor Growth

Methods

Cells

ZR-75: hormone-dependent cancer (ductal carcinoma) with oestrogen receptors (ATCC, USA is depository)

PC-3: adenocarcinoma (grade IV) with bone metastasis (ATCC, USA is depository).

A) ZR-75-1 human breast cancer: ZR-75-1 human breast cancer cells obtained from the American Tissue Culture Collection (Rockville, Md.) were cultured in phenol red-free RPMI 1640. The cells were supplemented with 2 mM L-glutamine, 1 mM Na-pyruvate, 100 IU penicillin/ml, 100 μg streptomycin/ml, and 10% (v/v) fetal bovine serum and incubated under a humidified atmosphere comprised of 95% air and 5% CO₂ at 37° C.

Female homozygous HSD nu/nu athymic mice (50 days old) were obtained from Harlan Sprague Dawley Inc. (Indianapolis, Ind.). Five mice were housed per vinyl cage, which was equipped with air filter lids and kept in laminar air flow hoods under pathogen-limiting conditions. The photoperiod was composed of a period of 14 h of light and a period of 10 h of darkness. Cages, bedding, and food (Agway Pro-Lab R-M-H diet #4018) were autoclaved prior to use. Water was acidified to pH of 2.8, autoclaved, and provided ad libitum. Bilateral ovariectomy (OVX) was performed on all animals one week prior to cell inoculation, under 2.5% isoflurane anesthesia mixed with oxygen. Simultaneously, an oestrogen (E₂) implant was inserted subcutaneously to stimulate initital tumor growth and appearance. E₂ implants were prepared in 1-cm long silastic tubing (inside diameter, 0.062 inch; outside diameter, 0.095 inch) containing 0.5 cm of estradiol/cholesterol diluted at a ratio of 1:10 (w:w). One week after the ovariectomy, 2.0×10⁶ ZR-75-1 cells, in their logarithmic growth phase, were harvested with 0.083% pancreatin/0.3 mM EDTA and inoculated s.c. in 0.1 ml of RPMI 1640 culture medium containing 30% of Matrigel, from each flank of each animal through a 2.5-cm-long 20-gauge needle. Four weeks after ZR-75-1 cell inoculation, the E₂ implants were replaced in all animals by estrone-containing implants (E₁: cholesterol; 1:25 w:w) (104). Treatments consisting of increased doses of bpV(phen) versus control were started 5 weeks after cell inoculation. Mice bearing tumors of an average area of 15 mm² were randomly assigned to 3 groups, each group containing more than 15 mice. OVX animals first received the most potent natural estrogen, to initiate cell proliferation and the development of tumors. Thereafter, the E₂ implants were replaced by E₁ implants as a model for post-menopausal women in which E₁ is the main circulating estrogen that is converted into E₂ in peripheral tissues.

On day 0 of the experiment (5 weeks after inoculation), the E₁-releasing implants were removed from the animals in Group 1 only. All mice received a daily administration of bpv(phen) over a period of 42 days (i.p., 100μl, in a 2 blind manner). Groups 1 and 2 received PBS, Group 3 received 2.5 mg/Kg bpV(phen).

B) Human prostate adenocarcinoma (PC-3) in the athymic mice: Male Balb/c nude (nu/nu) were purchased at 4-6 weeks of age from Charles Rivers Inc. Mice were housed under pathogen free conditions and maintained on a 12-h light/12-h dark cycle with food and water supplied ad libitum. The hormono-independent PC3 human tumor cells were from the American Tissue Cuture Collection. Cells were grown in DMEM in the presence of 5% foetal bovine serum. Cells were collected at confluence, included in a matrix (1.0×10⁶ cells/ml; 30% Matrigel). An equal volume of the tumor cell suspension was injected s.c. in the right flank of each mouse. After 5 days, a palpable tumor of approximately 5×5 mm was detected in the inoculated animals. Mice with palpable tumors were divided into five groups (18 mice/group) for the treatment study. All mice in each treatment group had tumor of similar size at the start of treatment. For administration to mice, bpV(phen) was dissolved and diluted in phosphate buffered saline (PBS) at pH 7.4. A 5 mg/Kg dose of bpV(phen) was administed daily by i.p. for 39 days. Taxol was used as a positive control at the dose of 20 mg/Kg and injected i.p. at every three days. A control group of 10 animals was injected with PBS. The injection volume was kept constant at 100 μl/g body weight. The mice were weighed three times during the experimental period to assess toxicity of the treatment, and the tumors were measured twice weekly using calipers. Tumor volume was calculated from the two-dimensional caliper measurements using the following formula: tumor volume=length×(width)²×0.53.

The treatment period was completed after 39 days, when the PBS treated group of mice had large tumors, requiring that the animals be sacrificed according to the Animal Care Procedures. On the final day of the study, the mice were sacrificed by carbon dioxyde inhalation. The s.c. tumors was removed and weighed.

Statistical analysis: Tumor growth curves are presented in terms of treatment group means and SEs. Statistical significance of treatment effect was assessed by repeated measures ANOVA after applying a power transformation to equalize residual variances and linearize the tumor growth curves.

Results

1. Progression of Tumor Cells in vitro

The cells embedded in the collagen gel, grew as a “primary tumor”. Some cells migrated from the primary tumor towards the fibrin gel, forming front edges. Small clumps of cells were observed in the fibrin gel; in this model they represent “secondary tumors”. Their extension and numbers are representative of the invasive potential of the cancer cells.

PC-3: In control experiments, PC-3 cells migrated slightly from the primary tumor and formed extensive secondary tumors in the fibrin gel. In the presence of 2 μM bpV(phen), a decrease in the size of the secondary tumor was observed and the migration front from the primary tumor was similar to that seen in the control gels. In the presence of 5 and 10 μM bpV(phen), there was no migration front and there were no secondary tumors in the fibrin gel. In addition, the primary tumors appeared clearer than in the control (FIG. 11).

ZR-75-1: In control experiments ZR-75-1 cells migrated into the fibrin as small spheroidal secondary tumors, with a limited and sparsely visible migration front. The presence of 2 μM bpV(phen) restricted the growth of the primary tumor. At 5 and 10 μM bpV(phen) there were no secondary tumors and the primary tumor had a lower cell density (FIG. 12).

2. Inhibition of Tumor Progression in vivo

A-ZR-75-1 Human Breast Cancer:

The tumor size in the control group, having not received E₁ replacement therapy, did not increase. The tumor size in the animals in the other control groups having received E₁ replacement therapy was found to have increased significantly (p<0.05) from 15 to 26 mm² on day 42. The daily administration of bpV(phen) do not resulted in increase of tumor size (p<0.05). The results show that bpV(phen) has the capacity to inhibit the progression of tumors in vivo (FIG. 13A).

B-Human Prostate Adenocarcinoma (PC-3) in the Athymic Mice:

Daily administration of bpV(phen) caused a significant (p<0.001) 59% suppression of the final tumor compared with PBS-treated control animals (FIG. 13B). No death were observed among the vehicle-treated controls or bpV(phen), and, on average these mice gained 1.5 and 1.7 grams in body weight respectively, relative to their weight at the initiation of the treatment.

F) The use of Inhibitors of Protein Tyrosine Phosphatases (PTP) for Anti-Tumor Immunotherapy

Lymphocytes with anti-tumor activity can be isolated from patients and grown in vitro for use in cell-tranfer therapies (119). The incubation of immune cells with the PTP inhibitor bpv(phen) augments their activation state (120). Therefore, the re-administration of bpV(phen)-activated immune cells to cancer patients may enhance the immune response towards tumor cells. The results described below demonstrate the efficacy of a method in which a peroxometallic compound (bpV(phen) is used ex vivo on autologous immune cells in order to stimulate the potency of these cells and once returned into blood circulation of cancer patients fight invasion malignant cells.

Method

An in vitro cancer invasion system that has been previously designed was used. Briefly, prostate cancer cells (PC-3; American Type Culture Collection, Rockville Md.) were grown in DMEM medium with 5% fetal bovine serum, 2 mM L-glutamine, and antibiotics. They were, incubated under a humidified atmosphere of 95% air/5% CO₂ at 37° C. Collagen gels containing the PC-3 cells were prepared according the method of Esdale and Bard (121). The cell-embedded collagen gels were laid down onto a layer of fibrin gel, and anchored by a second layer of fibrin gel. The top of the collagen gel was not fully covered with fibrin gel in order to allow direct contacts between the cancer cells in collagen and the splenocytes. The latter were directly seeded onto the top layer of the collagen and fibrin gel. Prior to the molding of the cancer invasion system, leucocytes were isolated from spleen of either healthy mice or mice bearing PC-3 tumors. They were treated in vitro with bpV(phen) (25 μM) for 24 hr. Thereafter, treated cells were washed, counted and seeded (10⁶ cells per gel) on the cancer cells-embedded gels. Untreated leukocytes seeded on the top of the cancer invasion system (same concentration) were used as a control experiment. Medium was renewed periodically. During the whole experiment, most leucocytes remained on the top of the gels, and have a normal morphology. Cell behavior was periodically observed for 7 days of culture, then recorded (by photography).

Results

Clumps of PC3 cells progressively appeared in the fibrin gel representing the invasive potential of the cancer cells. In the control 3D culture system, PC-3 cells migrated slightly from the primary site and formed extensive secondary tumors in the fibrin gel as described in previous studies (122, 123). In contrast to this, in the presence of bpV(phen)-treated leucocytes, neither secondary tumors nor migration front was observed. In addition, the primary tumors appeared less dense than in the control, indicating a smaller number of growing cells (FIG. 14).

Conclusion

The above describes a method consisting in the ex vivo autologous activation by bpV(phen) of leucocytes and their potential to trigger a cellular immune response against cancer cells. This may prove to be beneficial for several ex vivo technologies. For example: alone or in combination with interleukins and chemokines that trigger immune response to specific antigens or mutated cell types (124).

Although the present invention has been described by way of preferred embodiments thereof, these embodiments can be modified at will, within the scope of the appended claims, without departing from the spirit and nature of the subject invention.

### LIST OF REFERENCES

1. Posner, B. I., Faure, R., Burgess, J. W., Bevan, A. P., Lachance, D., Zhan-Sun, G., Fantus, I. G., Ng, J. B., Hall, D. A., Lum, B. S. & Shaver, A. (1994). Peroxovanadium compounds: a new class of potent phosphotyrosine-phosphatase inhibitors which are insulin mimetics. J. Biol. Chem. 269: 4596-4604.

2. Faure, R., Vincent, M., Dufour, M., Shaver, A. & Posner, B. (1995). Arrest at the G2/M transition of the cell-cycle by protein-tyrosine phosphatase inhibition: Studies on a neuronal and a glial cell line. J. Cell. Biochem. 58: 389-401.

3. Tonks, N. K. and B. G. Heel Combinatorial control of the specificity of PTPs. Current opinion in cell Biology 2001 13(2): 182-195.

4. Huyer et al. (1997)

5. Reydi, M. A. & Schwartz, S. M. (1981) Endothelial regeneration: Time course of initial changes after small defined injury to rat aortic endothelium. Lab. Invest. 44: 301-308.

6. Schwartz, S. M. & Liaw, L. (1993) Growth control and morphogenesis in the development of pathology of arteries. J. Cardiovasc. Pharmacol. 21 (suppl): S31-S49.

7. Auerback, R. (1994). Angiogenesis inhibition: a review. Pharmac. Ther. 63: 265-311.

8. Folkman, J. (1995). Angiogenesis in cancer, vascular, rhumatoid and other diseases. Nature Med. 1: 27-31.

9. Schreiber, A. B., Winkler, M. E. & Derynck, R. (1986). Transforming growth factor-alpha: a more potent angiogenic factor than epidermal growth factor. Science 232: 1250-1254.

10. Roberts, A. B., Sporn, M. B., Assoian, R. K., Smith, J. M., Roche, N. J. & Wakefield, L. M. (1986). Transforming growth factor type beta: rapid induction of fibrosis and angiogenesis in vivo and stimulation of collagen formation in vitro. Proc. Natl. Acad. Sci. USA 84: 4167-4171.

11. Fràter-Schröder, M., Risau, W., Hallmann, R., Gautschi, R. & Bohlen, P. (1987). Tumor necrosis factor type-alpha, a potent inhibitor of endothelial cell growth in vitro, is angiogenic in vivo. Proc. Natl. Acad. Sci. USA 84: 5277-5282.

12. Fett, J. W., Strydom, D. J., Lobb, R. R., Alderman, R. M., Riordn, J. F. & Vallee, B. L. (1985). Isolation and characterization of angiogenin, an angiogenic protein from human carcinoma cells. Biochemistry 24: 5480-5488.

13. Ziche, M., Jones, J. & Gullino, P. (1982). Role of prostaglandin E1 and copper in angiogenesis. J. Natl. Cancer Inst. 69: 475-482.

14. Dobson, D. E., Kambe, A., Block, E., Dion, T., Lu, H. & Castellot, Jr. J. J., Speigelman, B. M. (1990). 1-Butyrylglycerol: a novel angiogenesis factor secreted by differentiating adipocytes. Cell 61: 223-230.

15. Baird, A. & Durkin, T. (1986). Inhibition of endothelial cell proliferation by type-beta transforming growth factor: interaction with acidic and basic fibroblast growth factors. Biochem. Biophys. Res. Commun. 138: 476-482.

16. Fràter-Schröder, M., Muller, G., Birchmeier, W. & Bohlen, P. (1986). Transforming growth factor-beta inhibits endothelial cell proliferation. Biochem. Biophys. Res. Commun. 137: 295-302.

17. Folkman, J., & Klagsbrun, M. (1987). Angiogenic factors. Science 235: 442-447.

18. Klagsbrun, M. & D'Amore, P. A. (1991). Regulators of angiogenesis. Annu. Rev. Physiol. 53: 217-232.

19. Gospodarowicz, D., Ferrara, N., Schweigerer, L. & Neufeld, G. (1987). Structural characterization and biological functions of fibroblast growth factor. Endocr. Rev. 8: 95-104.

20. Thomas, K., Rios Candelore, M., Gimenez-Gallego, G., Di Salvo, J., Bennet, C., Rodkey, J. & Fizpatrick, S. (1985). Pure brain-derived acidic fibroblast growth factor is a potent vascular endothelial cell mitogen with sequence homology to interleukin-1. Proc. Natl. Acad. Sci. USA 82: 6409-6416.

21. Miyazono, K., Okabe, T., Urabe, A., Takata, F. & Heldin, C. -H. (1987). Purification and properties of an endothelial cell mitogen from human platelets. J. Biol. Chem. 262: 4098-4104.

22. Ishikawa, F., Miyazono, K., Hellman, U., Drexler, H., Westerndt, C., Hagiwara, K., Usuki, K., Takaku, F., Risau, W. & Heldin, C. -H. (1989). Identification of angiogenic activity and the cloning and expression of platelet-derived endothelial cell growth factor. Nature 338: 557-561.

23. Leung, D. W., Cachianes, G., Kuang, W. -J., Goeddel, D. V. & Ferrara, N. (1989). Vascular endothelial growth factor is a secreted angiogenic mitogen. Science 246: 1306-1312.

24. Houck, K. A., Ferrara, N., Winer, J., Cachianes, G., Li, B. & Leung, D. W. (1991). The vascular endothelial growth factor family: Identification of a fourth molecular species and characterization of alternative splicing of RNA. Mol. Endocrinol. 5: 1806-1814.

25. Breier, G., Albrecht, U., Sterrer, S. & Risau, W. (1992). Expression of vascular endothelial growth factor during embryonic angiogenesis and endothelial cell differentiation. Development 114: 521-532.

26. Bussolino, F., Albini, A., Garnussi, G., Presta, M., Viglietto, G., Ziche, M. & Persico, G. (1996). Role of soluble mediators in angiogenesis. Eur. J. Cancer 32A: 2401-2412.

27. Colville-Nash, P. R. & Willoughby, D. A. (1997). Growth factors in angiogenesis: current interest and therapeutic potential. Mol. Med. Today. 3: 14-23.

28. Knighton, D. R., Hunt, T. K., Scheuenstuhl, H., Halliday, B. J., Werb, Z. & Banda, M. J. (1983). Oxygen tension regulates the expression of angiogenesis factor by macrophages. Science 221: 1283-1285.

29. Ladoux, A. & Frelin, C. (1993). Hypoxia is a strong inducer of vascular endothelial growth factor mRNA expression in the heart. Biochem. Biophys. Res. Commun. 195:1005-1010.

30. Nomura, M., Yamagishi, S. I., Harada, S. I., Hayashi Y., Yarnashima, T., Yamashita, J. & Yamamoto H. (1995). Possible participation of autocrine and paracrine vascular endothelial growth factors in hypoxia-induced proliferation of endothelial cells and pericytes. J. Biol. Chem. 270: 28316-28324.

31. Walgenbach, K. J., Gratas, C., Shestak, K. C. & Becker, D. (1995). Ischaemia-induced expression of bFGF in normal skeletal muscle: a potential paracrine mechanism for mediating angiogenesis in ischaemic skeletal muscle. Nature Med. 1: 453-459.

32. Lew, A. P., Lew, N. S., Iliopoulos, O., Jiang, C., Kaeling Jr., W. G. & Goldberg, M. A. (1997). Regulation of vascular endothelial growth factor by hypoxia and its modulation by the von Hippel-Lindau tumor suppressor gene. Kidney Int. 51: 575-578.

33. Sandner, P., Wolf, K., Bergmaier, U., Gess, B. & Kurtz, A. (1997). Induction of VEGF and VEGF receptor gene expression by hypoxia: Divergent regulation in vivo and in vitro. Kidney Int. 51: 448-453.

34. Ferrara, N. (1995). Missing link in angiogenesis. Nature 376: 467.

35. Koch, A. E., Halloran, M. M., Haskell, C. J., Shaw, M. R. & Polyerini, P. J. (1995). Angiogenesis mediated by soluble forms of E-selectin and vascular cell adhesion molecule-1. Nature 376: 517-519.

36. Polyerini, J. P., Cotran, R. S., Gimbrone Jr, M. A. & Unanue, E. R. (1977). Activated macrophages induce vascular proliferation. Nature 269: 804-806.

37. Sunderkotter, C., Steinbrink, K., Goebeler, M., Bhardwaj, R. & Sorg, C. (1994). Macrophages and angiogenesis. J. Leukocyte Biol. 55: 410-422.

38. Polyerini, P. J. (1996). How the extracellular matrix & macrophages contribute to angiogenesis-dependant diseases. Eur. J. Cancer 14: 2430-2437.

39. Gross, J. L., Moscatelli, D., Jaffe, E. A. & Rifkin, D. B. (1982). Plasminogen activator and collagenase production by cultured capillary endothelial cells. J. Cell Biol. 95: 974-981.

40. Moscatelli, D. A., Rifkin, D. B. & Jaffe, E. A. (1985). Production of latent collagenase by human umbilical vein endothelial cells in response to angiogenic preparations. Exp. Cell Res. 156: 379-390.

41. Ingber, D. E. (1991). Extracellular matrix and cell shape: potential control points for inhibition of angiogenesis. J. Cell. Biochem. 47: 236-241.

42. Ingber, D. E. (1992). Extracellular matrix as a solid-state regulator in angiogenesis: identification of new targets for anti-cancer therapy. Sem. Cancer Biol. 3: 57-63.

43. Bischoff, J. (1995). Approaches to studying cell adhesion molecules in angiogenesis. Trends Cell Biol. 5: 69-74.

44. Eisenstein, R. (1991). Angiogenesis in arteries: review. Pharmac. Ther. 49: 1-19.

45. Folkman, J. & Ingber, D. (1992). Inhibition of angiogenesis. Cancer Biol. 3: 89-96.

46. Bicknell, R. (1994). Vascular targeting and the inhibition of angiogenesis. Ann. Oncol. 5: S45-S50.

47. Montesano, R., Pepper, M. S., Belin, D., Vassalli, J. -D. & Orci, L. (1988). Induction of angiogenesis in vitro by vanadate, an inhibitor of phosphotyrosine phosphatases. J. Cell. Physiol. 134: 460-466.

48. Montesano, R. & Orci, L. (1985). Tumor-promoting phorbol esters induce angiogenesis in vitro. Cell 42: 469-477.

49. Montesano, R., Vassalli, J. D., Baird, A., Guillemin, R. & Orci, L. (1986). Basic fibroblast growth factor induces angiogenesis in vitro. Proc. Natl. Acad. Sci. USA. 83: 7297-7301.

50. Sefton, B. M. & Hunter, T. (1984). Tyrosine protein kinases. Adv. Cyclic Nucleotide Protein Phosphorylation Res. 18: 195-203.

51. Hunter, T. & Cooper, J. A. (1985). Protein-tyrosine kinases. Annu. Rev. Biochem. 54: 897-930.

52. Hunter, T. & Cooper, J. A. (1986). Viral oncogenes and tyrosine phosphorylation. In: The Enzymes, Vol. 17. P. D. Boyer and E. G. Krebs, eds. Academic Press, Orlando, Fla., pp.191-246.

53. Ramachandran, J. & Ulirich, A. (1987). Hormonal regulation of protein tyrosine kinase activity. Trends Pharmacol. Sci. 8: 28-31.

54. Battistini, B., Chailler, P., D'Orlèans, J., Brière, N. & Sirois, P. (1993). Growth regulatory properties of endothelins. Peptides 14: 385-399.

55. Pedram, A., Razandi, M., Hu R-M & Levin, E. R. (1997). Vasoactive peptides modulate vascular endothelial cell growth factor production and endothelial cell proliferation and invasion. J. Biol. Chem. 272: 17097-17013.

56. Asham, E. H., Loizidou, M. & Taylor, I. (1998). Endothelin-1 and tumor development. Eur. J. Surg. Oncol. 24: 57-59.

57. Kusuhara, M., Yamaguchi, K., Nagasaki, K., Hayashi, C., Suzaki, A., Nori, S., Hanada, S., Nakamura, Y. and Abe, K. (1990). Production of endothelin in human cancer cell lines. Cancer Res. 50: 3257-3261.

58. Schichiri, M., Hirata, Y., Nakajiroa, T., & o, K., Imal, T., Yanagisawa, M., Mazaki, T. and Marumo, F. (1991). Endothelin-1 is an autocrine/paracrine growth factor for human cancer cell lines. J. Clin. Invest. 87: 1867-1871.

59. Pagotto, U., Arzberger, T., Hopfer, U., Weidnl, A. & Stella, G. (1995). Cellular localization of endothelin receptor mRNA ET_{A} and ET_{B} in the brain tumor and normal human brain. J. Cardiovasc. Pharmacol. 26 (Suppl. 3): S104-106.

60. Stiles, J. D., Ostrow, P. T., Balos, L. L., Greenberg, S. J., Plunkett, R., Grand, W. & Heffner Jr., R. R. (1997). Correlation of endothelin-I and transforming growth factor β1 with malignancy and vascularity in human gliomas. J. Neurobiol. 56: 435-439.

61. Ishibashi, M., Fujita, M., Nagai, K., Koko, M., Furue, H., Haku, E., Osamura, Y. & Yamaji, T. (1993). Production and secretion of endothelin by hepatocellular carcinoma. J. Clin. Endocrinol. Metab. 76: 378-383.

62. Giaid, A., Hamid, Q. A., Springall, D. R., Yanagisawa, M., Shinmi, O., Sawamura, T., Masaki, T., Kimura, S., Corrin, B. and Polak, J. M. (1990). Detection of endothelin immunoreactivity and mRNA in pulmonary tumors. J. Pathol. 162: 15-22.

63. Nelson, J., Chan, K., Hedican, S., Magnuson, S., Opgenorth, T., Bova, G. & Simons, J. (1996). Endothelin-1 production decreased B receptor expression in advanced prostate cancer. Cancer Res. 56: 663-668.

64. Kikuchik, K., Nakagawa, H., Kadono, T., Etoh, T., Byers, H., Mihm, M. & Tamaki, K. (1996). Decreased ETB receptor expression in human metastatic melanoma cells. Biochem. Biophys. Res. Commun. 219: 734-739.

65. Ziche, M., Morbidelli, L., Donnini, S. & Ledda, F. (1995). ETB receptor promote proliferation and migration of endothelial cells. J. Cardiovasc. Pharmacol. 26 (Suppl. 3): S284-S286.

66. Inagaki, H., Bishop, A., Escrig, C., Wharton, J., Allen-Mersh, T. & Polak, J. (1991). Localization of endothelin like immunoreactivity and endothelin binding sites in human colon. Gastroenterology 101: 47-54.

67. Inagaki, H., Bishop, A., Eimoto, T. & Polak, J. (1992). Autoradiographic localization of endothelin-1 binding sites in human colonic cancer tissue. J. Pathol. 168: 263-267.

68. Eguchi, S., Hirata, Y., Imai, T. & Marumo, P. (1995). ET-1 as an autocrine growth factor for endothelial cells. J. Cardiovasc. Pharmacol. 26 (Suppl. 3): S279-S283.

69. Leveson, S., Wiggins, P., Giles, G., Parkin, A. & Robinson, P. (1985). Deranged blood flow patterns in the detection of liver metastases. Br. J. Surg. 72: 128-130.

70. Carter, R., Anderson, J., Cooke, T., Baxter, J. & Angerson, W. (1994). Splanchnic blood flow changes in the presence of hepatic tumors, evidence of humoral mediator. Br. J. Cancer 69: 1025-1026.

71. Nakamura, T., Ebihara, I., Fukui, M., Tomino, Y. & Koide, H. (1995). Effect of a specific endothelin receptor A antagonist on mRNA levels for extracellular matrix components and growth factors in diabetic glomeruli. Diabetes 44: 895-899.

72. Lee, K. M., Tsai, K. Y., Wang, N. & Ingber, D. E. (1998). Extracellular matrix and pulmonary hypertension: control of vascular smooth muscle cell contractility. Am. J. Physiol. 274: H76-82.

73. Hirshfeld, J. W. Jr, Schwartz, J. S., Jugo, R., MacDonald, R. G., Goldberg, S., Savage, M. P., Bass, T. A., Vetrovec, G., Cowley, M., Taussig, A. S., et al. (1991) Restenosis after coronary angioplasty: a multivariate statistical model to relate lesion and procedure variables to restenosis. The M-HEART Investigators. J. Am. Coll. Cardiol. 18: 647-656.

74. Maseri, A. (1996). An unresolved, persisting challenge for percutaneous transluminal coronary angioplasty: how to identify the lesions that will not restenose. Eur. Heart J. 17: 814-815.

75. Kirchengast, M. & Munter, K. (1998). Endothelin and restenosis. Cardiovasc. Res. 39: 550-555.

76. Kramer, B. K., Ackermann, M., Kohler, S. M. & Riegger, G. A. (1994). Role of endothelin in hypertension. Clin. Investig. 72: 88-93.

77. Levin, E. R. (1995). Endothelins: mechanisms of disease. New Eng. J. Med. 333: 356-363.

78. Rohmeiss, P., Birck, R., Braun, C., Kirchengast, M. & van der Woude, F. J. (1999). Targets for endothelin in the diseased kidney: clues for therapeutic intervention. Exp. Nephrol. 7: 1-10.

79. Cardell, L. O., Uddman, R. & Edvinsson, L. (1994). Endothelins: a role in cerebrovascular disease? Cephalalgia 14: 259-65.

80. Cosentino, F. & Katusic, Z. S. (1994). Does endothelin-1 play a role in the pathogenesis of cerebral vasospasm? Stroke 25: 904-908.

81. Battistini, B. & Dussault, P. (1998). The many aspects of endothelins in ischemia-reperfusion injury: emergence of a key mediator. J. Invest. Surg. 11: 297-313.

82. Mauel, J. (1990). Macrophage-parasite interactions in Leishmania infections. J. Leukoc. Biol. 47: 187-193.

83. Belosevic, M., Findbloom, D. S., Meltzer, M. S. & Nacy, C. A. (1990). IL-2. A cofactor for induction of activated macrophage resistance to infection. J. Immunol. 145: 831-839.

84. Olivier, M., Bertrand, S. & Tanner, C. E. (1989). Killing of Leishmania donovani by activated liver macrophages from resistant and susceptible strains mice. Int. J. Parasitol. 19: 377-383.

85. Skov, C. B. 8 Twohy, W. (1974). Cellular immunity to Leishmania donovani. I. The effect of T cell depletion on resistance to L. donovani in mice. J. Immunol. 113: 2004-2011.

86. Murray, H. W., Masur, H. & Keithly, J. S. (1982). Cell-mediated immune response in experimental visceral leishmaniasis. I. Correlation between resistance to Leishmania donovani and lymphokine-generating capacity. J. Immunol. 129: 344-350.

87. Olivier, M. & Tanner, C. (1989). The effect of cyclosporin A in murine visceral leishmaniasis. Trop. Med. Parasitol. 40: 32-38.

88. Reiner, N. E. & Finke, J. H. (1983). Interleukin 2 deficiency in murine leishmaniasis donovani and its relationship to depressed spleen cell responsees to phytohemagglutinin. J. Immunol. 131: 1487-1491.

89. Murray, H. W., Stem, J. J., Welte, K., Rubin, B. Y., Carriero, S. M. & Nathan, C. F. (1987). Experimental visceral leishmaniasis: production of interleukin 2 and interferon-gamma, tissue immune reaction, and response to treatment with interleukin 2 and interferon-gamma. J. Immunol. 138: 2290-2297.

90. Olivier, M., Prouix, C. & Tanner, C. E. (1989). Importance of lymphokines in the control of the multiplication and dispersion of Leishmania donovani within liver macrophages of resistant and susceptible mice. J. Parasitol. 75: 720-727.

91. Green, S. P., Hamilton, J. A. & Phillips, W. A. (1992). Zymosan-triggered tyrosine phosphorylation in mouse bone-marrow-derived macrophages is enhanced by respiratory-burst priming agents. Biochem. J. 288: 427-432.

92. Greenberg, S., Chang. P. & Silverstein, S. C. (1993). Tyrosine phosphorylation of the gamma subunit of Fc gamma receptors, p72syk, and paxillin during Fc receptor-mediated phagocytosis in macrophages. J. Biol. Chem. 269: 3897-3902.

93. Dong, Z., Qi, X., Xie, K. & Fidler, I. J. (1993). Protein tyrosine kinase inhibitors decrease induction of nitric oxide synthase activity in lipopolysaccharide-responsive and lipopolysaccharide-non responsive murine macrohages. J. Immunol. 151: 2717-2724.

94. Dong, Z., O'Brian, C. A. & Fidler (1993). I. J., Activation of tumoricidal properties in macrophages by lipopolysaccharide requires protein-tyrosine kinase activity. J. Leukoc. Biol. 53: 53-60.

95. Glaser, K. B., Sung, A., Bauer, J. & Weichman, B. M. (1993). Regulation of eicosanoid biosynthesis in the macrophage. Involvement of protein tyrosine phosphorylation and modulation by selective protein tyrosine kinase inhibitors. Biochem. Pharmacol. 45: 711-721.

96. Golden, A., Nemeth, S. P. & Brugge, J. S. (1986). Blood platelets express high levels of the pp6oc-src-specific tyrosine kinase activity. Proc. Natl. Acad. Sci. USA 83: 852-856.

97. Golden, A. & Brugge, J. S. (1989). Thrombin treatment induces rapid changes in tyrosine phosphorylation in platelets. Proc. Natl. Acad. Sci. USA 86: 901-905.

98. Olivier, M, Romero-Gallo B. J., St-Laurent, R., Racette, N., Stevenson, M., Jacobs, P., Posner, B. I., Tremblay, M., & Faure, R. (1998). Modulation of INFg induced macrophage activation by phosphotyrosine phosphatase inhibition. J. Biol. Chem. 273: 13944-13949.

99. Chao, W., Liu, H., Hanahan, D. J. & Olson, M. S. (1992). Protein tyrosine phosphorylation and regulation of the receptor for platelet-activating factor in rat Kupffer cells. Effect of sodium vanadate. Biochem. J. 288: 777-784.

100. Secrist, J. P., Burns, L. A., Karnitz, L., Koretsky, G. A. & Abraham, R. T. (1993). Stimulatory effects of the protein tyrosine phosphatase inhibitor, pervanadate, on T-cell activation events. J. Biol. Chem. 268: 5886-5893.

101. Imbert, V., Peyron, J. F., Farahi Far, D., Mari, B., Auberger, P. & Rossi, B. (1994). Induction of tyrosine phosphorylation and T-cell activation by vanadate peroxide, an inhibitor of protein tyrosine phosphatase. Biochem. J. 297: 163-173.

102. Barbeau, B., Bernier, R., Dumais, N., Briand, G., Olivier, M., Faure, R., Posner, B. I. & Tremblay, M. (1997). Activation of HIV-1 LTR transcription and virus replication via NF-? B-dependent and -independent pathways by potent phosphotyrosine phosphatase inhibitors, the peroxovanadium compounds. J. Biol. Chem. 272: 12968-12977.

103. Janvier, R., Sourla, A., Koutsilieris, M. & Doillon, C. (1997). Stromal Fibroblasts are required for PC-3 human prostate cancer cells to produce capillary-like formation of endothelial cells in a three-dimensional co-culture system. Anticancer res. 17: 1551-1558.

104. Couillard S, Gutman M, Labrie F, Candos B, Labrie C. (1999). Effect of combined treatment with EM-800 and radiotherapy on the growth of human 212-75-1 breast cancer xenografts in nude mice. Cancer Res.59: 4857-4863.

105. Sirois, E., Côté, M. F. & Doillon, C. J. (1993). Growth factors and biological supports for endothelial cell lining: in vitro study. Int. J. Artificial Organs 16: 609-619.

106. Nicosia, R. F. & Ottineti, A. (1990) Growth of microvessels in serum-free-matrix culture of aorta. A quantitative assay of angiogenesis in vitro. Lab. Invest., 63:115-122.

107. Passaniti, A., Taylor, R. M., Pili, R., Guo, Y., Long, P. V., Haney, J. A., Pauly, R. R., Grant, D. S. & Martin, G. R. (1992). A simple quantitative method for assessing angiogenesis and antiangiogenic agents using reconstituted basement membrane, heparin, and fibroblast growth factor. Lab. Invest. 67: 519-528.

108. Laporte, S., & Escher, E. (1992). Neointima formation after vascular injury is angiotensin II mediated. Biochem. Biophys. Res. Comm. 187:1510-1516.

109. Lima, G. M., Vallochi, A. L., Silva, U. R., Bevilacqua, E. M., Kiffer, M. M. & Abrahamsohn, I. A. (1998). The role of polymorphonuclear leukocytes in the resistance to cutaneous leishmaniasis. Immunol. Lett. 64: 145-151.

110. Hobbs, A. J., Higgs, A. & Moncada, S. (1999). Inhibition of nitric oxide synthase as a potential therapeutic target. Annu. Rev. Pharmacol. Toxicol. 39: 191-220.

111. Ajuevor, M. N., Virag, L., Flower, R. J., Perretti, M. & Szabo, C. (1998).

Role of inducible nitric oxide synthase in the regulation of neutrophil migration in zymosan-induced inflammation. Immunology. 95: 625-630.

112. Yan, X. T., Tumpey, T. M., Kunkel, S. L., Oakes, J. E. & Lausch, R. N. (1998). Role of MIP-2 in neutrophil migration and tissue injury in the herpes simplex virus-1-infected cornea. Invest. Ophthalmol. Vis. Sci. 39:1854-1862.

113. Duong, M., Ouellet, N., Simard, M., Bergeron, Y., Olivier, M. & Bergeron M. G. B. (1998). Kinetic study of host defense and inflammatory response to Aspergillus fumigatus in steroid-induced immunosuppressed mice. J. Infect. Dis. 178: 1472-1482.

114. Tam, F. W., Karkar, A. M., Smith, J., Yoshimura, T., Steinkasserer, Kurrle, R., Langner, K. & Rees, A. J. (1996) Differential expression of macrophage inflammatory protein-2 and monocyte chemoattractant protein-1 in experimental glomerulonephritis. Kidney Int. 49: 715-721.

115. Stenger, S., Thuring, H., Röllinghoff, M. & Bogdan, C. (1994). Tissue expression of inducible nitric oxide synthase is closely associated with resistance to Leishmania major. J. Exp. Med. 180: 783-793.

116. Stenger, S., Donhauser, N., Thuring, H., Röllinghoff, M. & Bogdan, C. (1996). Reactivation of latent leishmaniasis by inhibition of inducible nitric oxide synthase. J. Exp. Med. 183: 1501-1514.

117. Pani, G., Kozlowski, M., Cambier, J., Mills, G. B. & Siminovitch, K. A. (1995) Identification of the tyrosine phosphatase PTP1C as a B cell antigen receptor-associated protein involved in the regulation of B cell signaling. J. Exp. Med. 181: 2077-2084.

118. Klingmüller, U., Lorenz, U., Cantley, L. C., Neel, B. G. & Lodish, H. F. (1995). Specific recruitment of SH-PTP1 to the erythropoietin receptor causes inactivation of JAK2 and termination of proliferative signals. Cell 80: 729-738.

119. Rosenberg, S. A. Progress in human tumor immunology and immunotherapy. Nature 411: 380-384. 2001.

120. Olivier, M. Romero-Gallo B. J., St Laurent, R., Racette, N., Stevenson, M., Jacobs, P., Posner, B., Tremblay, M., Faure, R. Modulation of INFg-induced macrophage activation by phosphotyrosine phosphatase inhibition. J. Biol. Chem. 273,13944-13949 (1998).

121. Esdale, T., Bard, J. Collagen substrata for studies on cell behavior. J. Cell. Biol. 54: 626-637 (1972).

122. Janvier, R., Sourla, A., Koutsilieris, M., Doillon, C. Stromal fibroblasts are required for PC-3 human prostate cancer cells to produce capillary -like formation of endothelial cells in a three-dimensional co-culture system. Anticancer Res. 17: 1551-1557 (1997).

123. Dvorak, H. F., Harvey, V. S., Estrella, P., Brown, L. F., McDonagh, J., Dvorak A. M. Fibrin containing gels induce angiogenesis. Implications for stroma generartion and wound healing. Lab Invest. 57: 673-686 (1987).

124. Eberlein, T. J., Rosenstein, M. & Rosenberg S. A. Regression of a disseminated syngeneic solid tumor by systemic transfer of lymphoid cells expanded in interleukin 2. J. Exp. Med. 56: 385-397 (1982).

What is claimed is:

1. A method for preventing or arresting angiogenesis in an animal comprising administering a therapeutically effective amount of a compound of the following formula to prevent said angiogenesis: 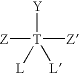
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

2. A method as defined in claim 1, wherein said angiogenesis is related to arthritis, psoriasis, a disorder of the eye or a condition characterized by the development of solid tumors.

3. A composition for preventing or arresting in an animal comprising a pharmaceutically-acceptable excipient and a therapeutically effective amount of a compound of the following formula to prevent said angiogenesis: 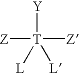
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

4. A composition as defined in claim 3, wherein said angiogenesis is related to arthritis, psoriasis, a disorder of the eye or a condition characterized by the development of solid tumors.

5. A device comprising a composition as defined in claim 3.

6. A method for preventing or lessening endothelin production in an animal by inhibiting protein tyrosine phosphatases (PTPs) comprising administering a therapeutically effective amount of a compound of the following formula: 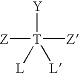
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

7. A composition for preventing or lessening endothelin production in an animal by inhibiting protein tyrosine phosphatases (PTPS) comprising a pharmaceutically-acceptable excipient and a therapeutically effective amount of a compound of the following formula: 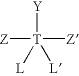
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

8. A method for preventing restenosis following angioplasty in an animal comprising administering a therapeutically effective amount of a compound of the following formula: 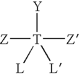
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

9. A composition for preventing restenosis following angioplasty in an animal comprising a pharmaceutically-acceptable excipient and a therapeutically effective amount of a compound of the following formula: 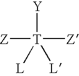
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

10. A device comprising a composition as defined in claim 9.

11. A device as defined in claim 10, wherein said device is a stent.

12. A method for modulating inflammation and secretion of inflammatory molecules in an animal comprising administering a therapeutically effective amount of a compound of the following formula: 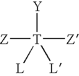
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

13. A composition for modulating inflammation and secretion of inflammatory molecules in an animal comprising a pharmaceutically-acceptable excipient and a therapeutically effective amount of a compound of the following formula: 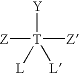
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

14. A device comprising a composition as defined in claim 13.

15. A method for inducing the activation of cytokines and chemokines in an animal comprising administering a therapeutically effective amount of a compound of the following formula: 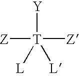
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

16. A method as defined in claim 15, wherein said cytokines are IL-12, IFN-γ, IL-1α or IL-1β and said chemokines are RANTES, MIP-1α, MIP-1β, MIP-2, IP-10 or MCP-1.

17. A composition for inducing the activation of cytokines and chemokines in an animal comprising a pharmaceutically-acceptable excipient and a therapeutically effective amount of a compound of the following formula: 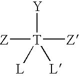
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

18. A composition as defined in claim 17, wherein said cytokines are IL-12, IFN-γ, IL-1α or IL-1β and said chemokines are RANTES, MIP-1α, MIP-1β, MIP-2, IP-10 or MCP-1.

19. An adjuvant having the following formula: 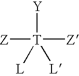
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

20. A vaccine comprising an adjuvant as defined in claim 19.

21. A vaccine as defined in claim 20, wherein said vaccine is against Leishmania parasite or other pathogens.

22. A method for preventing or arresting tumor growth in an animal comprising administering a therapeutically effective amount of a compound of the following formula: 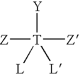
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

23. A composition for preventing or arresting tumor growth in an animal comprising a pharmaceutically-acceptable excipient and a therapeutically effective amount of a compound of the following formula: 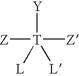
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

24. A device comprising a composition as defined in claim 23.

25. A method for treating cancer in an animal comprising administering a therapeutically effective amount of a compound of the following formula: 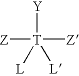
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

26. A composition for treating cancer in an animal comprising a pharmaceutically-acceptable excipient and a therapeutically effective amount of a compound of the following formula: 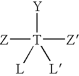
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

27. A device comprising a composition as defined in claim 26.

28. A method as defined in claim 25, wherein said cancer is breast cancer or prostate cancer.

29. A composition as defined in claim 26, wherein said cancer is breast cancer or prostate cancer.

30. A vaccine against cancer comprising a compound of the following formula: 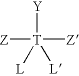
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

31. A method for activating leucocytes in an animal comprising administering to said animal leucocytes that have been treated in vitro with a compound of the following formula: 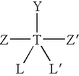
wherein
T is a transition metal selected from the group consisting of vanadium, molybdenum, tungsten, titanium, nobium and tantalum,
Y is oxygen or hydroxyl,
Z and Z′ are independently selected from oxygen and peroxide and at least one of them is peroxide, and
L or L′ are any group which can donate one electron pair.

32. A method as defined in claim 31 for use in the treatment of cancer.

33. A method as defined in claim 1, wherein T is vanadium.

34. A method as defined in claim 1, wherein T is molybdenum.

35. A method as defined in claim 1, wherein T is tungsten.

36. A method as defined in claim 1, wherein Z is oxygen and Z′ is peroxide.

37. A method as defined in claim 1, wherein Z and Z′ are peroxide.

38. A composition as defined in claim 3, wherein T is vanadium.

39. A composition as defined in claim 3, wherein T is molybdenum.

40.A composition as defined in claim 3, wherein T is tungsten.

41. A composition as defined in claim 3, wherein Z is oxygen and Z′ is peroxide.

42. A composition as defined in claim 3, wherein Z and Z′ are peroxide.

43. A method as defined in claim 8, wherein T is vanadium.

44.A method as defined in claim 8, wherein T is molybdenum.

45. A method as defined in claim 8, wherein T is tungsten.

46. A method as defined in claim 8, wherein Z is oxygen and Z′ is peroxide.

47. A method as defined in claim 8, wherein Z and Z′ are peroxide.

48. A composition as defined in claim 9, wherein T is vanadium.

49.A composition as defined in claim 9, wherein T is molybdenum.

50. A composition as defined in claim 9, wherein T is tungsten.

51. A composition as defined in claim 9, wherein Z is oxygen and Z′ is peroxide.

52. A composition as defined in claim 9, wherein Z and Z′ are peroxide.

53.A device comprising a composition as defined in claim 48.

54. A device comprising a composition as defined in claim 49.

55. A device comprising a composition as defined in claim 50.

56. A device comprising a composition as defined in claim 51.

57. A device comprising a composition as defined in claim 52.

58.A device as defined in claim 53, wherein said device is a stent.

59. A device as defined in claim 54, wherein said device is a stent.

60. A device as defined in claim 55, wherein said device is a stent.

61. A device as defined in claim 56, wherein said device is a stent.

62. A device as defined in claim 57, wherein said device is a stent.

63.An adjuvant as defined in claim 19, wherein T is vanadium.

64. An adjuvant as defined in claim 19, wherein T is molybdenum.

65. An adjuvant as defined in claim 19, wherein T is tungsten.

66. An adjuvant as defined in claim 19, wherein Z is oxygen and Z′ is peroxide.

67. An adjuvant as defined in claim 19, wherein Z and Z′ are peroxide.

68. A vaccine comprising an adjuvant as defined in claim 63.

69. A vaccine comprising an adjuvant as defined in claim 64.

70. A vaccine comprising an adjuvant as defined in claim 65.

71. A vaccine comprising an adjuvant as defined in claim 66.

72. A vaccine comprising an adjuvant as defined in claim 67.

73. A method as defined in claim 22, wherein T is vanadium.

74. A method as defined in claim 22, wherein T is molybdenum.

75. A method as defined in claim 22, wherein T is tungsten.

76. A method as defined in claim 22, wherein Z is oxygen and Z′ is peroxide.

77. A method as defined in claim 22, wherein Z and Z′ are peroxide.

78. A composition as defined in claim 23, wherein T is vanadium.

79. A composition as defined in claim 23, wherein T is molybdenum.

80. A composition as defined in claim 23, wherein T is tungsten.

81. A composition as defined in claim 23, wherein Z is oxygen and Z′ is peroxide.

82. A composition as defined in claim 23, wherein Z and Z′ are peroxide.

83. A device comprising a composition as defined in claim 78.

84. A device comprising a composition as defined in claim 79.

85. A device comprising a composition as defined in claim 80.

86. A device comprising a composition as defined in claim 81.

87. A device comprising a composition as defined in claim 82.

88. A method as defined in claim 25, wherein T is vanadium.

89. A method as defined in claim 25, wherein T is molybdenum.

90. A method as defined in claim 25, wherein T is tungsten.

91. A method as defined in claim 25, wherein Z is oxygen and Z′ is peroxide.

92. A method as defined in claim 25, wherein Z and Z′ are peroxide.

93. A composition as defined in claim 26, wherein T is vanadium.

94. A composition as defined in claim 26, wherein T is molybdenum.

95. A composition as defined in claim 26, wherein T is tungsten.

96. A composition as defined in claim 26, wherein Z is oxygen and Z′ is peroxide.

97. A composition as defined in claim 26, wherein Z and Z′ are peroxide.

98. A device comprising a composition as defined in claim 93.

99. A device comprising a composition as defined in claim 94.

100. A device comprising a composition as defined in claim 95.

101. A device comprising a composition as defined in claim 96.

102. A device comprising a composition as defined in claim 97.

103. A vaccine as defined in claim 30, wherein T is vanadium.

104. A vaccine as defined in claim 30, wherein T is molybdenum.

105. A vaccine as defined in claim 30, wherein T is tungsten.

106. A vaccine as defined in claim 30, wherein Z is oxygen and Z′ is peroxide.

107. A vaccine as defined in claim 30, wherein Z and Z′ are peroxide.

108. A method as defined in claim 31, wherein T is vanadium.

109. A method as defined in claim 31, wherein T is molybdenum.

110. A method as defined in claim 31, wherein T is tungsten.

111. A method as defined in claim 31, wherein Z is oxygen and Z′ is peroxide.

112. A method as defined in claim 31, wherein Z and Z′ are peroxide.

113. A method as defined in claim 32, wherein T is vanadium.

114. A method as defined in claim 32, wherein T is molybdenum.

115. A method as defined in claim 32, wherein T is tungsten.

116. A method as defined in claim 32, wherein Z is oxygen and Z′ is peroxide.

117. A method as defined in claim 32, wherein Z and Z′ are peroxide.

118. A method as defined in claim 1, wherein said compound is bpV(phen), bpV(pic) or bpV(bipy).

119. A composition as defined in claim 3, wherein said compound is bpV(phen), bpV(pic) or bpV(bipy).

120. A method as defined in claim 8, wherein said compound is bpV(phen), bpV(pic) or bpV(bipy).

121. A composition as defined in claim 9, wherein said compound is bpV(phen), bpV(pic) or bpV(bipy).

122. A device comprising a composition as defined in claim 121.

123. A device as defined in claim 122, wherein said device is a stent.

124. An adjuvant as defined in claim 19, wherein said adjuvant is bpV(phen), bpV(pic) or bpV(bipy).

125. A vaccine comprising an adjuvant as defined in claim 124.

126. A vaccine as defined in claim 125, wherein said vaccine is against Leishmania parasite or other pathogens.

127. A method as defined in claim 22, wherein wherein said compound is bpV(phen), bpV(pic) or bpV(bipy).

128. A composition as defined in claim 23, wherein said compound is bpV(phen), bpV(pic) or bpV(bipy).

129. A device comprising a composition as defined in claim 128.

130. A method as defined in claim 25, wherein wherein said compound is bpV(phen), bpV(pic) or bpV(bipy).

131. A composition as defined in claim 26, wherein said compound is bpV(phen), bpV(pic) or bpV(bipy).

132. A device comprising a composition as defined in claim 131.

133. A vaccine as defined in claim 30, wherein said compound is bpV(phen), bpV(pic) or bpV(bipy).

134. A method as defined in claim 31, wherein said compound is bpV(phen), bpV(pic) or bpV(bipy).

135. A method as defined in claim 32, wherein said compound is bpV(phen), bpv(pic) or bpV(bipy).
